(19) United States
(12) Patent Application Publication
(10) Pub. No.: US 2025/0255570 A1
(43) Pub. Date: Aug. 14, 2025

(54) SYSTEM AND METHOD FOR IN VIVO TISSUE IMAGING USING CODED APERTURE X-RAY SCATTER TOMOGRAPHY

(71) Applicant: CALIDAR, INC., dURHAM, NC (US)

(72) Inventors: Joshua Howard CARPENTER, Raleigh, NC (US); Stefan Matthias STRYKER, Durham, NC (US)

(21) Appl. No.: 18/856,954

(22) PCT Filed: Apr. 14, 2023
(86) PCT No.: PCT/US23/18682

§ 371 Date: Oct. 15, 2024

Related U.S. Application Data
(60) Provisional application No. 63/331,764, filed on Apr. 15, 2022.

(51) Int. Cl. A61B 6/00; A61B 6/06; A61B 6/42

(52) U.S. Cl. CPC A61B 6/5282; A61B 6/06; A61B 6/4233; A61B 6/545; A61B 6/4441

(57) ABSTRACT

A system and method for in vivo tomographic tissue imaging using coded aperture X-ray scatter tomography are disclosed. The imaging system includes a coded aperture for spatially encoding X-ray scatter originating from within a patient. An X-ray detector array records the modulated scatter signal, which is analyzed and used to generate a spatially resolved X-ray scatter spectral reconstruction of the tissue and produce a spatially resolved scatter tissue image of the irradiated tissue.

### CROSS-REFERENCE TO RELATED APPLICATION

This application claims the benefit of priority to U.S. Provisional Patent Application No. 63/331,764, entitled “System and Method for In Vivo Tissue Imaging Using Coded Aperture X-Ray Scatter Tomography.” which was filed on Apr. 15, 2022, the entire contents of which is incorporated by reference herein in its entirety.

### TECHNICAL FIELD

The present invention relates to medical imaging and x-ray scatter tomography, and more specifically, to a system and method for in vivo tissue imaging using coded aperture X-ray scatter tomography.

### BACKGROUND

In vivo noninvasive volumetric imaging techniques allow clinicians to create internal 3D images of patients. Four techniques in particular-X-ray transmission computed tomography (transmission CT), magnetic resonance imaging (MRI), positron emission tomography (PET), and ultrasound (US)-arc commonly used by clinicians to make a variety of medical assessments. These existing techniques all suffer from poor tissue contrast in a variety of applications, particularly applications which require discrimination between regions of soft tissue with varying properties. Cancer detection applications are one such class of applications, which require the ability to discriminate between regions of cancerous and benign soft tissue. All existing in vivo volumetric tissue imaging techniques require clinicians to have extensive training and experience in order to make assessments on the malignancy of regions of imaged tissue while misclassification rates for cancer detection still leave significant room for improvement, even for the best clinicians using any of these existing techniques. There are also significant costs to the health care system, worse health outcomes for patients, and unnecessary added stress for patients due to misclassifications in cancer detection applications. Therefore, there is a need for an in vivo noninvasive volumetric imaging technique that can better discriminate tissue types, particularly for regions of soft tissue and for cancer detection applications.

### SUMMARY

According to one embodiment of the present invention, a spatially resolved volumetric tissue imaging system for performing in vivo imaging of a patient body is disclosed. The imaging system includes an enclosure comprising a bore and a gantry positioned around the bore, wherein the bore is configured to receive at least a portion of the human body. The imaging system further includes an X-ray source for irradiating in vivo tissue of at least a portion of the body with a primary X-ray beam. The X-ray source is mounted to the gantry, and the X-ray source is configurable to change the orientation of the primary X-ray beam about the bore axis and the exposure time. The imaging system further includes a collimator positioned between the X-ray source and the tissue to shape the primary X-ray beam. The imaging system further includes an X-ray detector array comprising an arrangement of X-ray detecting elements in at least two dimensions. A plurality of the X-ray detecting elements are positioned distally from the X-ray source outside the path of the primary X-ray beam past the irradiated portion of the body to measure scattered X-ray radiation from the primary X-ray beam passing through the tissue. The imaging system further includes a coded aperture positioned between the tissue and the X-ray detector array. The coded aperture is configured to modulate the scattered X-ray radiation from the tissue detected by the X-ray detector array. The imaging system further includes a control system comprising memory and a processor. The processor is configured for configuring the imaging system for performing an X-ray scatter measurement based on configuration data comprising the orientation of the primary beam relative to the bore axis and exposure time for the X-ray source. The processor is further configured for performing the X-ray scatter measurement with the configured imaging system. The processor is further configured for receiving data representing scattered X-ray radiation detected by the X-ray detector array. The processor is further configured for estimating a spatially resolved X-ray scatter spectral reconstruction of the tissue based on the received X-ray scatter data and the configuration data. The processor is further configured for determining a spatially resolved tissue property based on the received X-ray scatter data. The processor is further configured for producing a spatially resolved scatter tissue image based on the received X-ray scatter data.

According to another embodiment, an imaging system for performing in vivo imaging of a human body is disclosed. The imaging system includes an X-ray source mounted to a configurable arm for irradiating in vivo tissue of at least a portion of the body with a primary X-ray beam. The position and orientation of the X-ray source is adjustable by the user. The imaging system further includes a collimator positioned between the X-ray source and the tissue to shape the primary X-ray beam. The imaging system further includes an X-ray detector array comprising a plurality of X-ray detecting elements arranged in at least two dimensions. The at least one of the X-ray detecting elements is positioned distally from the X-ray source outside the path of the primary X-ray beam past the irradiated portion of the body to measure scattered X-ray radiation from the primary X-ray beam passing through the tissue. The imaging system further includes a coded aperture positioned between the tissue and the X-ray detector array. The coded aperture is configured to modulate the scattered X-ray radiation from the tissue detected by the X-ray detector array. The imaging system further includes a control system comprising memory and a processor. The processor is configured for determining configuration data for the imaging system. The configuration data comprises the location and orientation of the X-ray source. The processor is further configured for performing the X-ray scatter measurement with the configured imaging system. The processor is further configured for receiving data representing scattered X-ray radiation detected by the X-ray detector array. The processor is further configured for estimating a spatially resolved X-ray scatter spectral reconstruction of the tissue based on the received X-ray scatter data and the configuration data. The processor is further configured for determining a spatially resolved tissue property based on the received X-ray scatter data. The processor is further configured for producing a spatially resolved scatter tissue image based on the received X-ray scatter data.

According to another embodiment, a method for performing in vivo tissue imaging of a patient body is disclosed. The method includes positioning at least a portion of the body in the imaging system. The method further includes configuring the imaging system for an X-ray scatter measurement based on configuration data comprising the orientation of the primary beam relative to the bore axis and exposure time for an X-ray source. The method further includes performing the X-ray scatter measurement with the configured imaging system. The X-ray scatter measurement includes irradiating in vivo tissue of at least a portion of the body with a primary X-ray beam from the X-ray source through a collimator positioned between the X-ray source and the tissue to shape the primary X-ray beam. The X-ray scatter measurement further includes modulating scattered X-ray radiation from the tissue using a coded aperture positioned between the tissue and an X-ray detector array comprising an arrangement of X-ray detecting elements in at least two dimensions. The X-ray scatter measurement further includes detecting the modulated scattered X-ray radiation signal from the tissue with a plurality of the X-ray detecting elements positioned distally from the X-ray source outside the path of the primary X-ray beam past the irradiated portion of the body to measure scattered X-ray radiation from the primary X-ray beam passing through the tissue. The X-ray scatter measurement further includes receiving data representing the detected scattered X-ray radiation from the X-ray detector array. The X-ray scatter measurement further includes estimating a spatially resolved X-ray scatter spectral reconstruction of the tissue based on the received X-ray scatter data and the configuration data. The X-ray scatter measurement further includes determining a spatially resolved tissue property based on the received X-ray scatter data. The X-ray scatter measurement further includes producing a spatially resolved scatter tissue image based on the received X-ray scatter data.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure, and may be more generally applied.

### BRIEF DESCRIPTION OF THE DRAWINGS

The disclosed invention will be better understood by reading the following non-limiting descriptions of embodiments, that reference the attached drawings, in which:

FIG. 1 depicts a schematic of a CT system with coded aperture for scatter measurements, where the coded aperture and detector can be within the gantry enclosure or within the CT scanner bore according to an embodiment of the subject matter described herein.

FIG. 2 depicts a flowchart for conducting CT transmission and scatter acquisition by the system according to an embodiment of the subject matter described herein.

FIG. 3 depicts a schematic of a coded aperture and how the coded aperture can be flat or curved relative to the oncoming scattered X-rays according to an embodiment of the subject matter described herein.

FIG. 4 depicts a schematic of a CT system with motorized mechanism for moving coded aperture into and out of the beam path for transmission vs scatter measurements modes according to an embodiment of the subject matter described herein.

FIG. 5 depicts a schematic of a CT system showing how a primary pencil/fan beam can enter the patient from any angle while the coded aperture moves with the detector for performing measurements according to an embodiment of the subject matter described herein.

FIG. 6 depicts a schematic of a CT system showing angled source-side collimation for converting a fan beam to pencil beam for measuring scatter through target regions of a patient according to an embodiment of the subject matter described herein.

FIG. 7 depicts a schematic of a CT with coded aperture and extra detector built into a gantry outside of the primary X-ray beam path according to an embodiment of the subject matter described herein.

FIG. 8 depicts a schematic of a CT system with separate X-ray sources and detectors at different tunnel depth locations for transmission and scatter measurements according to an embodiment of the subject matter described herein.

FIG. 9 depicts a schematic of a CT scanner having dual sources and detectors for transmission and scatter measurements within the same plane according to an embodiment of the subject matter described herein.

FIG. 10 depicts a schematic illustrating accounting for patient motion during scatter measurements according to an embodiment of the subject matter described herein.

FIG. 11 depicts a schematic of a CT system using a highly focused coded aperture near the detector for scatter measurement of single point along a pencil beam path according to an embodiment of the subject matter described herein.

FIG. 12 depicts a schematic of raster scanning of the pencil beam and highly focused coded aperture shown in FIG. 11 to measure multiple voxels according to an embodiment of the subject matter described herein.

FIG. 13 depicts a schematic of a C-arm X-ray transmission system combined with a coded aperture for scatter measurements, where the coded aperture can be mounted to the system and moved to an optimal measurement position according to an embodiment of the subject matter described herein.

FIG. 14 depicts a schematic of a ceiling-mounted X-ray transmission system combined with a coded aperture for scatter measurements, where the coded aperture can be placed in air or within a patient table for encoding of scatter data according to an embodiment of the subject matter described herein.

FIG. 15 depicts a schematic of a portable X-ray transmission system combined with a coded aperture for scatter measurements, where the coded aperture and detector can be placed in conjunction with the portable X-ray system for measurements according to an embodiment of the subject matter described herein.

FIG. 16 depicts a schematic of a general CT system with an arrow passing through the bore of the gantry to indicate the axis that is referred to as the bore axis or patient motion axis according to an embodiment of the subject matter described herein.

FIG. 17 depicts a schematic of a CT bore and gantry system with a coded aperture built into a table for the purpose of receiving and scatter imaging the breast of a patient during breast CT imaging according to an embodiment of the subject matter described herein.

### DETAILED DESCRIPTION

The subject matter described herein includes a system, method, and control system for tomographic X-ray scatter imaging of tissue in vivo. The present disclosure can be used to acquire spatially resolved, volumetric tissue property estimates of in vivo tissue.

As used herein, the phrase “reconstructing an image” is not intended to exclude embodiments of the present invention in which data representing an image is generated but a viewable image is not. Therefore, as used herein, the term “image” broadly refers to both viewable images and data representing a viewable image. However, many embodiments generate (or are configured to generate) at least one viewable image.

In the descriptions herein, descriptions including but not limited to the measured scatter signal and reconstructed spatially resolved scatter spectra arc referred to as pertaining to the “X-ray scatter”, though the X-ray scatter field will in general be comprised of both Rayleigh and Compton scatter, also known as coherent and incoherent scatter. The use of the terms “scatter” or “diffraction” in these descriptions throughout are not intended to limit the present invention to pertaining to scatter arising from one physical process and not another.

X-ray transmission computed tomography (transmission CT), magnetic resonance imaging (MRI), and positron emission tomography (PET) are commonly used existing in vivo volumetric imaging techniques used by clinicians to make a variety of medical assessments. In these techniques, electromagnetic radiation is detected once it has passed through or been emitted from within the body.

While the specifics of the acquired data depend on the technique, in general, detectors are used to quantify signal intensity and frequency as a function of the time and location that the signal was measured. Intensity or frequency modulations in space or time of the measured signals may result from the physical mechanism each technique is based upon (e.g., X-ray attenuation for transmission CT), and the protocols of the measurement type (e.g., using shorter repetition times and times-to-echo in T1-weighted MRI scans). The raw measured data for any of these techniques are complex and multidimensional, such that they are not of any immediate diagnostic value to a clinician. Specialized data processing and algorithmic techniques are chosen to reconstruct spatially resolved tissue data that are of value to the clinician from the modulations in the raw data.

Two factors are used in determining the diagnostic value of the images generated by in vivo imaging techniques to a clinician: spatial resolution and image contrast. Increasing spatial resolution allows the clinician to identify smaller features and better resolve tissue feature shape. Image contrast in this context refers to variations in intensity in the spatially resolved reconstructed data and is determined by the specific protocols of the particular measurement type, the choice of data processing procedure and reconstruction algorithm used to generate the image, and the underlying physics of the technique, the last of which sets fundamental limits on what can be quantified from the raw data. The value of image contrast to the clinician depends on the application. Preferably the measurement technique, data processing, and reconstruction algorithm are chosen such that they maximize the contrast between the tissue types and features that are most useful to the clinician in making a particular assessment. Because reconstructed image contrast is associated with the physical mechanism each technique is based upon, transmission CT, MRI, and PET imaging have associated strengths and weaknesses depending on the types of tissue to be differentiated by the clinician in a given application.

One example of image-contrast enhancement is the development of various novel contrast-enhancing dyes for both MRI and transmission CT, which can preferentially increase contrast of certain tissue types. An example technique designed to enhance tissue contrast is dynamic contrast enhanced MRI, which can detect differences in microvasculature through variations in spatially resolved reconstructed intensities over time in a series of sequential measurements.

While application-specific image contrast is important to generating useful images for clinicians, there is more information in the raw measured data for each of these techniques than is typically quantified in the reconstructed tissue images used by clinicians. In general, these techniques have the potential to generate spatially resolved spectral tissue maps, i.e., the reconstructed spatial maps can have an additional spectral dimension in each voxel, rather than scalar values, which allows for differing, potentially application specific, contrast in each spectral bin. Some methods for acquiring spectral tissue maps include combining techniques as in PET/CT or PET/MRI imaging, conducting multiple measurements with different parameters as in T1-weighted and T2-weighted MRI scans or in transmission CT scans with varying X-ray source filtration, or conducting multiple measurements with varying levels or types of contrast enhancing dyes using MRI or transmission CT. Some specialized variations of the techniques also allow for spatially resolved spectral tissue maps in a single measurement, such as hyperspectral transmission CT, which uses energy discriminating X-ray detectors to quantify tissue voxel radiodensity in multiple energy ranges, and magnetic resonance spectroscopic imaging which quantifies the magnetic resonance spectrum in each tissue voxel.

In vivo imaging techniques are used by clinicians in cancer detection applications such as cancer screening, diagnosis, and staging, as well as directing and monitoring treatment such as radiation therapy, to make shape and contrast-based assessments of regions of tissue maps as cancerous or benign. Most cancer detection applications require tissue discrimination of regions of soft tissue and, despite significant advancements in transmission CT, MRI, and PET methods, soft tissue contrast is generally weak, particularly between cancerous and benign tissue. Using any of these techniques requires extensive clinician training and experience to make accurate classifications. With enhanced contrast between soft tissue types, cancer classification performance could be significantly improved across all cancer detection applications. Advancements that increase tissue contrast, particularly for cancerous versus benign soft tissue, or that provide spatially resolved spectral information to clinicians to aid in their assessments of in vivo images, fill an unmet need and may significantly improve expected patient outcomes and lower costs.

X-ray scatter tomography is another noninvasive imaging technique that can be used to generate spatially resolved volumetric spectral maps of material that has, to date, not been implemented in vivo for tissue imaging. More specifically, tomographic X-ray scatter imaging modalities can generate spatially resolved volumetric maps of X-ray scatter spectra. For tomographic X-ray diffraction imaging modalities in particular, spatially resolved volumetric maps of material momentum transfer spectra can be reconstructed. Momentum transfer spectral data are inaccessible with existing in vivo imaging techniques, such as transmission CT, MRI, or PET. Momentum transfer spectra reflect local molecular ordering and can have numerous distinct features for each material, such that they are particularly suited for differentiating materials. An in vivo implementation of X-ray scatter tomography for medical imaging applications can provide a clinician with volumetric tissue images that generated in an application-specific manner from the reconstructed spectral maps, with contrast chosen based on specific known features that differentiate the most relevant types of tissue, such as cancerous and benign tissue. Such images, with tissue type contrast generated from features in the momentum transfer spectra of a specific tissue type, would never be accessible with existing imaging techniques.

Components of the in vivo imaging systems and methods described herein can vary between embodiments. A non-exhaustive description of such variations is described below. Variations in the X-ray source include but are not limited to the type of source (e.g. X-ray generator versus radioactive isotope), generator anode material, and generator focal spot size. Variations in the collimator include varying collimator opening size and shape to control the spatial extent and cross-sectional shape, respectively, of the initial X-ray beam. These aspects of the collimator opening arc controllable with the control system in some embodiments of the system. Variations in coded aperture design include but are not limited to pattern types (e.g, periodic, Fresnel zone plate, random or optimized random, uniformly redundant array types), material, open fraction, and thickness. Systems may also include multiple coded apertures that can be chosen by the user or automatically based on measurement conditions, e.g., the optimal coded aperture may be selected algorithmically based on geometric constraints and estimated radiation dose for an X-ray scatter measurement on a user-specified subregion of tissue. Variations in the X-ray detector array include but are not limited to number of X-ray detectors that make up the array, number of detecting elements or pixels per detector, pixel size and pitch, detector and detector array dimensionality (e.g. 2D or 3D), detection mechanism (e.g. scintillator or direct conversion), absorption layer thickness, and energy or photon discriminability (e.g. energy integrating, stacked multi-energy channel, energy discriminating, or photon counting). While an energy integrating X-ray detector type, typical of most X-ray transmission imaging systems, may be used, an energy discriminating or photon counting detector may provide for improved signal-to-noise ratio, radiation dose reduction to the patient, and overall performance of the system. Larger or higher dimensional X-ray detector arrays may capture more of the scattered X-ray signal which may also allow for improved signal-to-noise ratio, radiation dose reduction to the patient, and overall performance of the system.

Embodiments of the in vivo systems and methods described herein include reconstructing spatially resolved volumetric X-ray spectral data from an irradiated volume of tissue using a coded aperture to modulate the scatter, an X-ray detector array comprising an arrangement of X-ray detecting elements in at least two dimensions to measure the modulated scatter signal, a forward matrix model of the physics and the geometry of the measurement configuration, and a processor to iteratively estimate the spatially resolved volumetric X-ray spectral data from the forward matrix model. Another embodiment further comprises estimating momentum transfer spectra on a pixel or voxel basis. Another embodiment further comprises using the spatially resolved momentum transfer spectral map in combination with a reference library of existing tissue momentum transfer spectra of known tissue types, to calculate spatially resolved estimates of tissue type of the irradiated volume of tissue. The reference momentum transfer spectra can specifically include those of cancerous or benign tissue types to calculate spatially resolved estimates of the likelihood of cancer.

An embodiment of the method described herein further includes optimizing the X-ray scatter measurement configuration by calculating the radiation dose to the patient and the estimated X-ray scatter data quality of the measurement. The method includes calculating an estimate of radiation dose to the patient for an X-ray scatter measurement from the configuration data, calculating an estimate of resulting X-ray scatter data quality metrics for the configuration data, and optimizing the configuration data using the estimated radiation dose and the estimated X-ray scatter data quality metrics. The optimized configuration data used by the processor enacts an X-ray scatter measurement optimized for estimated radiation dose and estimated X-ray scatter data quality metrics. Such embodiments include controlling various components of the system. One example is an embodiment wherein the X-ray source is an X-ray generator and the X-ray source current or X-ray source voltage is optimized. Another example is an embodiment wherein the collimator opening size or shape can be optimized. Another example is an embodiment wherein a moveable filter can be disposed in the initial X-ray beam to optimize the energy spectrum and irradiance of the beam. Another example is an embodiment wherein the relative location or orientation of at least one of the X-ray source, the collimator, the coded aperture, or a plurality of X-ray detecting elements can be optimized. Another example is an embodiment wherein the relative location of the tissue can be optimized. An important metric for the X-ray scatter data quality is the signal to noise ratio. The measured X-ray scatter signal will be significantly less intense than the transmitted X-ray signal for the same initial X-ray beam and irradiated volume and attenuation of the relatively weaker X-ray scatter signal by the tissue will impact the signal to noise ratio of the measured signal. An embodiment of the method described herein comprises configuring components to reduce the impact of the attenuation of the tissue on the measured X-ray scatter signal, specifically on the signal to noise ratio. This is achieved by selecting a perspective of the tissue by defining the initial X-ray beam path to minimize the attenuation of the initial X-ray beam along the initial X-ray beam path to a point in the tissue or to minimize the attenuation of the scattered X-ray signal from the point in the tissue to an X-ray detecting element. This can include minimizing path lengths or avoiding more attenuating paths, such as through bone.

The method and imaging system of the present invention provide for in vivo tissue imaging using single, few, or many perspectives of the patient, based on the configuration of X-ray sources, collimators, coded apertures, and X-ray detecting elements relative to the patient in a given embodiment. For embodiments that allow a plurality of perspectives of the patient, 3D or hyperspectral tissue maps can be generated tomographically from data acquired at each perspective. Incorporating multiple X-ray sources at different locations in the imaging system also allows for multiple perspectives of the patient in some embodiments. Additionally, moveable or rotatable X-ray sources and X-ray detector elements can be used to enable different perspectives of the patient in some embodiments. In some embodiments these moveable or rotatable components are controllable with the control system. For example, a gantry located around the patient on which the X-ray source and X-ray detecting elements can be mounted which can rotate about the patient and translate along the bore axis direction (i.e., longitudinal axis direction) in which the patient (or a portion of the patient) is located, can allow for any number of views of the patient from any angle. In embodiments in which the X-ray detecting elements and X-ray sources are moveable, measurements can be made while the key components are fixed for a single perspective, repeated at multiple fixed positions for multiple perspectives, or conducted while key components are in motion such that the perspective varies with time. In embodiments in which the collimator is moveable in a controllable manner with the processor, the collimator can be held at a fixed relative location and orientation between an X-ray source and the tissue, or moved independently of the X-ray source, such that the direction of the beam relative to the X-ray source, tissue, and X-ray detecting elements may be controlled for further diversity of imaging perspective. More specifications to the method and imaging system of this invention that facilitate integration with transmission CT imaging components and methods are described below, with accompanying Figures.

In considering a quality metric that could be computed by the disclosed invention to optimize the configuration for X-ray scatter measurements, there are a variety of properties that could be accounted for in optimizing the quality of the measurement. As examples, but not limitation, these factors could include the X-ray attenuation of the primary beam or scattered X-rays aiming to minimize dose to patient or maximize the signal to noise ratio (SNR) of the measured data. For quality metrics that consider the noise of a measurement, the optimization could focus on total noise in acquired data, the ratio of the noise to signal overall or within regions of the detected data, or the type of noise (e.g., Poisson, Gaussian) that will be present in the measurement. This quality metric could also account for orienting the measurement to limit the primary X-ray beam path or scattered X-rays emitted through organs where dose reduction is desired. Additionally, the quality metric could account for the location of patient bones that would absorb more of the primary or scattered X-rays, to avoid the reduction in signal or increase in scan time or dose that would be needed to overcome this additional absorption. Furthermore, if the embodiment of the invention is one that scatter scans only a sub-region of the patient to lower dose, the quality metric of the measurement could account for measuring multiple suspicious masses or regions of interest within a single pencil beam or fan beam measurement, where the illumination geometry is optimized to measure all desired regions using the fewest number of exposures necessary to achieve the task. Additionally, the quality metric for scatter imaging may account for spatial positioning of the region of interest (e.g. suspicious mass), by positioning the region closer to the coded aperture or X-ray source by controlling their positioning, to achieve a desired level of geometric magnifications of the scattered X-rays or coded aperture features at the detector. These provided examples suggest a variety of factors that may be included in a quality metric used to optimize imaging procedures, but the list is not exhaustive and other factors used and known within the field may be applied to the quality metric and measurement optimization process.

The components mounted on the gantry may be configured to move along an arbitrary curve rather than along the path of a circle. In some CT systems, the X-ray source and detector may be stationary while the object or patient is rotated; it should be appreciated that the descriptions contained herein are also applicable to such imaging methods.

In addition to allowing for multiple imaging perspectives and tomographic reconstructions, embodiments of the method in which the location and orientation of key components can be controlled present further advantages. Embodiments with filters that can be moved into or out of the initial beam allow for controlling the irradiance and energy spectrum. Similarly, collimators of varying shape (e.g., pencil, fan, cone, or annular), may be moved into or out of the beam to control the cross-sectional shape of the initial beam, while moving a collimator closer or further from the source focal spot allows for controlling beam divergence, both of which allow for control of the irradiated tissue volume and radiation dose to the patient. Controlling coded aperture location and orientation allows for optimizing the code magnification by varying the relative distances from points on the coded aperture to both the irradiated tissue volume and the X-ray detecting elements. Controlling location and orientation of X-ray detecting elements allows for optimizing for the range of momentum transfer space data that is collected, based on the relative location of the X-ray detecting elements to the irradiated tissue volume and the energy spectrum of the initial beam, as well as the momentum transfer space resolution based on detecting element or pixel size and relative location. In embodiments in which the X-ray source, collimator, coded aperture, or X-ray detecting elements are moveable, the configuration data further comprises relative locations or relative orientations of the moveable components. The calculation of the representation of the irradiated tissue is based on these configuration data. The accuracy of the system geometry used for calculations to generate the representation of the irradiated, particularly the accuracy of the coded aperture geometry, may limit the possible reconstructed spectral tissue map accuracy.

In another embodiment of the present invention, a full or half-circle ring of X-ray detectors surround the bore, as is typical of 4th and 5th generation transmission CT scanners, for measuring X-rays scattered from the primary beam or for use to measure the transmitted primary beam through the tissue, rather than other embodiments that employ a smaller detector with an X-ray source that both rotate on a gantry, as is typical of the 3rd generation design of transmission CT scanners. In another embodiment of the present invention the X-ray source rotates on a gantry while a ring of detectors is stationary around a patient. Furthermore, another embodiment utilizes a full ring or sections of detectors positioned around a bore or patient, with multiple X-ray sources positioned at multiple different orientations about the bore axis to enable multiple perspectives of the patient without any moving components. In this embodiment, the X-ray sources could be multiple radioactive sources with individual shutters that could be timed together for CT data collection, or X-ray generators including currently available models or those using new technologies like carbon nanotubes that could be positioned around the patient. These multiple X-ray sources and detectors could be used to collect multi-view data simultaneously or in a sequential manner.

Further advantages of the imaging system described herein may be associated with various embodiments that include a plurality of particular components. For example, embodiments that use multiple X-ray generators with different anode materials allow for initial X-ray beams with different peak energies. Embodiments with multiple filters allow for switching or combining filters to control the irradiance and energy spectrum of the initial X-ray beam. Embodiments with multiple collimators, particularly those that can be moved independently, allow for varying the spatial extent of the beam, the irradiated tissue volume, and the radiation dose to the patient. Embodiments with multiple coded apertures, particularly those in which the coded apertures can be moved independently, allow for switching one coded aperture for another with a different code pattern that better codes the measured scatter for a given measurement configuration (e.g., location and/or orientation) and the types of tissue the user is seeking to classify and discriminate between. Because the scattered X-ray signal is weaker than the transmitted X-ray signal, one advantage of embodiments with multiple X-ray detectors making up the X-ray detector array or a larger X-ray detector array in general is the ability to record as much of the X-ray scatter signal as possible. Embodiments in which a 3D X-ray detector array comprising multiple, separate, 2D X-ray detectors also allow for each detecting element or pixel to be more accurately oriented toward the irradiated tissue volume than does an X-ray detector array comprising a single 2D X-ray detector with the same total detector area. X-ray detector arrays that consist of curved X-ray detectors are another example of 3D X-ray detector arrays in which each detecting element or pixel can be more accurately oriented toward the irradiated tissue volume.

The method of the present invention can be paired with various other imaging techniques to take advantage of complementary strengths of the two techniques. One such embodiment of the present method, pairs tomographic X-ray scatter imaging with transmission X-ray imaging, which has the built-in advantage of also using X-ray hardware. Some such embodiments of the method take advantage of this by sharing the same X-ray sources, filters, collimators, or X-ray detecting elements for both X-ray scatter and X-ray transmission measurements.

While some embodiments of the present invention use an X-ray generator as an X-ray source, other embodiments utilize radioactive materials as an X-ray source, with some embodiments including both types of sources. In an embodiment with a radioactive source, radioactive material can be placed within an enclosure that absorbs X-rays and provides shielding with a window and shutter that can be utilized to control the X-rays exiting the window and produce the primary beam for irradiating the tissue and performing the scatter measurement. In such an embodiment, the exposure time is controlled by opening and closing the shutter rather than by turning the voltage and current going towards an anode on and off as is the case within an X-ray generator. Such an embodiment could be advantageous for scatter imaging by having a narrower or approximately monoenergetic energy spectrum of the primary beam relative to a broader energy spectrum generally representative of Bremsstrahlung produced within the anode of X-ray generators. As a few illustrative examples not meant to be limiting, Cobalt-57 (emits 136.6 keV X-rays, half-life of 270 days) or Americium-241 (emits 59.5 keV X-rays, half-life of 432 years) are radioactive isotopes that produce X-rays with energies that could be useful and acceptable for clinical applications of an embodiment of the disclosed invention. A narrower or approximately monoenergetic spectrum could reduce the complexity of the system model used to estimate the spatially resolved scatter reconstruction and by doing so reduce computational costs and increase accuracy. This could also allow for similarly accurate reconstructions with reduced dose to patients by reducing the number of photons irradiating the patient within specific energy ranges. Such an embodiment could require replacement of the radioactive source over time as the activity decays. In another embodiment, an X-ray generator is used for 2D or 3D X-ray transmission imaging, while a radioactive source could be utilized for the scatter imaging.

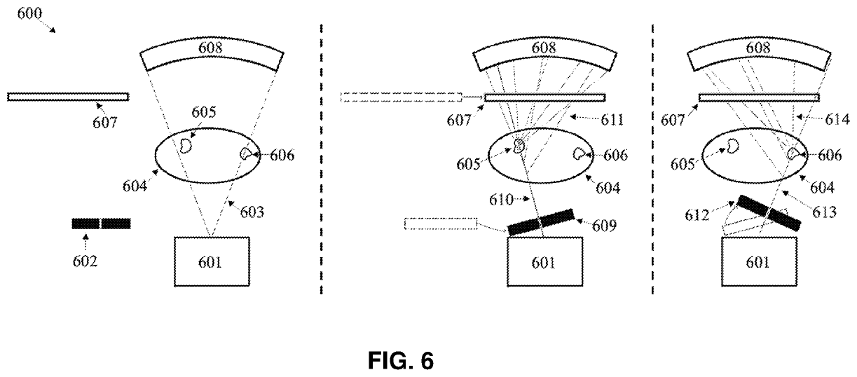

Specific embodiments of the present method that comprise both tomographic X-ray scatter imaging and X-ray transmission imaging can further comprise performing an X-ray transmission measurement on a volume of tissue followed by an X-ray scatter measurement on a specified subregion of the tissue volume, i.e. spot-checking regions from the initial X-ray transmission measurement with an X-ray scatter measurement. Such embodiments of the method have the advantage of decreased radiation dose relative to X-ray scatter measurements conducted on the entire volume of tissue. Such embodiments allow for spatially resolved tissue property estimates of subregions of tissue that are of concern to an operator, such as those that are suspected to be cancerous, while minimizing radiation dose to the patient required for the measurement. An X-ray scatter measurement on a subregion of tissue can be performed in various ways which depend on the specific embodiment. Some examples of steps to define a subregion for the X-ray scatter measurement include but are not limited to: using an additional collimator to change the spatial extent of the beam, moving the collimator to change the direction or divergence of the initial X-ray beam, varying the size of the collimator opening to change the spatial extent of the beam, moving the X-ray source or X-ray detecting elements to change the perspective of the tissue, and moving the tissue to change the perspective of the tissue. In some embodiments of the present method, the processor receives user input for the selection of the subregion of tissue for the X-ray scatter measurement. The processor calculates configuration data for the user input for at least one of the X-ray source, the collimator, the coded aperture, or a plurality of X-ray detecting elements and enacts the calculated configuration data with the components to perform the X-ray scatter measurement on the subregion of tissue. In another embodiment, the method further comprises transmitting a radiodensity tissue image, calculated from the X-ray transmission measurement, to a display and the user input further comprises an indication of the subregion in the displayed radiodensity tissue image. In another embodiment, the method further comprises computing a spatially resolved estimate of the likelihood of cancer from the radiodensity tissue image, computing regions of interest in the radiodensity tissue image using the spatially resolved estimate of the likelihood of cancer, and transmitting the region of interest data to the display. In another embodiment, the method further comprises using machine learning algorithms to compute the regions of interest in the radiodensity tissue image. Such embodiments with region of interest indicators help point out potentially cancerous regions to an operator, which they could choose to select as a subregion on which to perform an X-ray scatter measurement. A specific embodiment of the imaging system and method for X-ray scatter measurement spot-checking in a CT scanner system are discussed in more detail below and shown in FIG. 6.

Embodiments of the present method that include X-ray transmission imaging can also be advantageous because the X-ray transmission data may be used by the reconstruction algorithm to generate joint reconstruction estimates of tissue voxel radiodensity and X-ray scatter spectra. In such embodiments, the physics of the X-ray interactions with the illuminated tissue volume may already be contained in the forward matrix model of the system along with the geometry and relevant properties of any shared hardware between the imaging modalities. In other embodiments of the method, X-ray transmission data may be incorporated into calculations of the estimated tissue type, which may improve classification performance. Due to the advantages of embodiments of the method of the present invention which include transmission X-ray imaging, there are several specific embodiments which describe in more detail below how the imaging system described in the current method may be integrated into the general configuration of an in vivo transmission CT scanner using similar components.

There are a multitude of X-ray scatter properties, which are a subset of a tissue's properties, that can be measured, computed, or displayed to a user by the invention disclosed herein. For example, but not limitation, a reconstruction algorithm can utilize the measured scattered X-rays to compute momentum transfer spectra, a type of X-ray diffraction spectra that accounts for the energies of X-rays which impacts scattering angles, but the system could also compute an intensity vs scatter angle (or 20) profile that does not account for the energies of the X-rays produced by the X-ray source. From these scatter spectra of various types, additional properties can be computed including the total scatter intensity (summing intensity vs angle or momentum transfer), intensity-weighted average scatter angle or intensity-weighted average momentum transfer value, scatter intensity for sub-ranges of the scattering angles or momentum transfer q values, and ratios of scatter intensities for different spectral peaks or sub-ranges of the scattering angles or momentum transfer. These additional metrics can be utilized in classification algorithms for identifying/differentiating different tissue types or for prognostic applications. Additionally, the different scatter-based metrics can be utilized to generate images for viewing by a user, whether these images are composed purely of data from the scatter measurement or a combination of imaging data types. The combination images could include, but not limited to, CT/2D radiograph/MRI data colorized by the scatter properties, or the ability for a user to select a pixel/voxel/toxel of interest within the medical image and have information relating to the scatter properties reported. This reporting, particularly for regions with scatter properties that correspond with cancer or a condition of interest, could be automatically flagged or visually displayed to the user for case of identifying regions that may need further investigation.

On the topic of conditions of interest and tissue properties, while we highlight cancer in particular, there are a range of other tissue properties related to other diseases or biological conditions that the disclosed invention could be utilized for identifying or providing prognostic information. Beyond identifying if a suspicious mass within the body is malignant, other diseases like ductal carcinoma in situ (DCIS) within the breast is often referred to as pre-cancer, but there are many patients whose DCIS never progresses to invasive carcinoma. Measured scatter properties could be useful in identifying which patients have DCIS that will progress to cancer, and which can be observed for longer or denoted as a non-threat to the patient. The inflamed state of organs including the lungs during disease or infections could also potentially lead to a change in the scattering properties that could be measured by the disclosed invention and utilized for a range of clinical purposes. Additionally, changes in the liver during cirrhosis could lead to changes in X-ray scattering properties and represents another change in tissue property that the invention could be utilized to identify. Furthermore, it is possible that changes in the brain during the onset and progression of dementia, or specifically Alzheimer's disease, could lead to changes in the composition or structure of cells that could be measured by scattered X-ray properties. These examples, which are not limiting, of additional tissue properties and their related conditions/diseases are meant to illustrate the variety of useful properties that could be measured and reported by the disclosed invention.

Transmission CT scanners typically utilize a circular gantry within an enclosure that allows for rapid positioning of X-ray sources and X-ray detectors at various positions (both radially about the longitudinal axis and translationally in the direction of the longitudinal axis) relative to the objects being imaged. Components rotate on the gantry to capture data from multiple views, which allows 2D slices to be reconstructed from the data. A bore in CT scanners is a central opening having a bore axis or longitudinal axis, through which patients can be positioned on a table that moves them through the bore relative to the X-ray imaging system, allowing for multiple slice measurements or continuously acquired helical scan data, either of which allows for the reconstruction of a full 3D image.

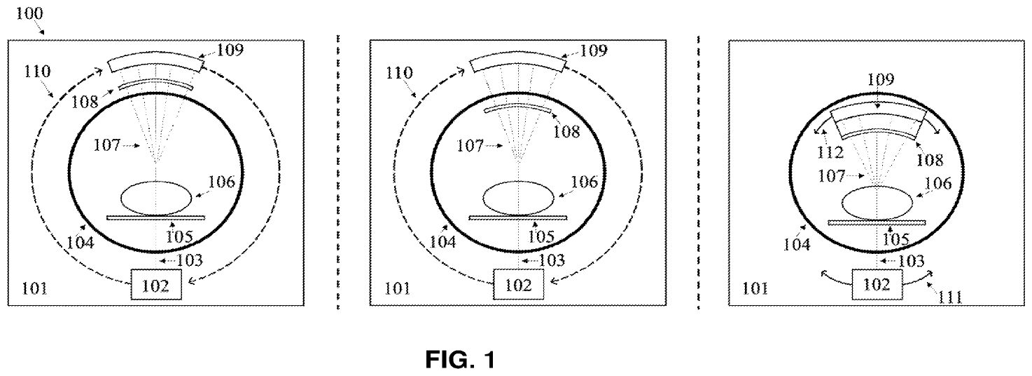

FIG. 1 depicts a schematic of a CT system with coded aperture for scatter measurements, where the coded aperture and X-ray detector can be within the gantry enclosure or within the CT scanner bore according to an embodiment of the subject matter described herein. FIG. 1 modifies the imaging architectures mentioned above for coded aperture scatter imaging and presents one view along the bore or longitudinal axis of the configuration where components of the imaging system can be positioned. Referring to FIG. 1, the imaging system 100 includes a gantry enclosure 101, X-ray source 102. X-ray primary beam 103, CT scanner bore 104, patient table 105, patient 106, X-ray scatter 107, coded aperture 108, X-ray detector 109, gantry 110 & 111 for rotating X-ray components, and non-CT gantry translation and rotation of an X-ray detector and coded aperture within the CT bore 112. The left panel of FIG. 1 shows how the coded aperture 108 may be built inside the gantry 110, moving with the X-ray detector 109 for scatter imaging from any perspective of the patient. The center panel of FIG. 1 is another configuration with the coded aperture 108 positioned within the region of the CT bore 104 (either in open air or as part of an enclosure that is within the typical bore location). The right panel of FIG. 1 shows an X-ray dctcctor 109 as well as a coded aperture 108 positioned within the CT bore 104. There are advantages for each of the three choices of the location of the coded aperture 108 and X-ray scatter detector 109. With both components outside the bore (as shown in the left panel of FIG. 1) attached to the gantry 110, the components do not take up any room inside the bore, to keep patient size limits the same as in existing CT scanners, and may rotate together on the gantry to maintain the same relative location. With the coded aperture 108 inside the bore (as shown in the center panel of FIG. 1), the coded aperture is closer to the irradiated tissue, allowing for more magnification of the coded scatter features onto the X-ray detector 109 still outside the bore. Using the X-ray scatter detector 109 inside the bore (as shown in the right panel of FIG. 1) provides higher signal levels because the bore wall material does not attenuate the scattered X-rays. This allows for improved reconstruction quality in less time and therefore may reduce the necessary radiation dose to the patient.

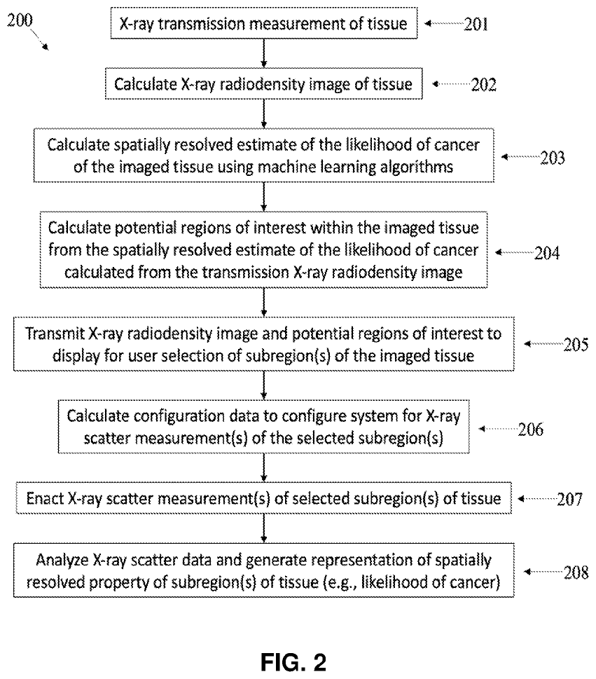

FIG. 2 depicts a flowchart of exemplary steps for conducting CT transmission and scatter acquisition by the system according to an embodiment of the subject matter described herein. Referring to FIG. 2, measurements begin with a transmission CT scan of the patient at step 201, which is used for computing the radiodensity image of the tissue in step 202. In step 203, a machine learning algorithm operates on the radiodensity image, producing the likelihood of cancer that can be used to calculate potential regions of interest in step 204, which a user can utilize in step 205 when selecting subregion(s) of interest for further scatter measurements, which may be used by the operator to highlight a region of interest for scatter measurements at step 202. After making this selection, the system configures for a scatter measurement mode at step 206, in which the processor converts the user input region of interest into system configuration data for an X-ray scatter measurement of the region of interest. The system configuration data can be further optimized by the processor to minimize radiation dose or maximize estimated X-ray scatter data quality metrics. The processor then implements the system configuration. This may include movement of key components. For example, the coded aperture may be moved into the region between the irradiated tissue and the X-ray detector if the same X-ray detector was being used for transmission and scatter measurements. Collimators may be moved relative to the source focal spot to change the initial beam direction or the total illuminated tissue volume. Separate X-ray components, such as an X-ray source and X-ray detector, may also be used for the scatter measurements. Alternatively, the same components may be used with different operating parameters. Other components that may be used for a scatter measurement but not used for a standard CT transmission measurement may also be moved into different positions, such as positioning a beam block to block the transmitted initial beam for the scatter measurement. Next, scatter measurements may be conducted at step 207, followed by post-processing of the scatter data at step 208. The post-processing generates tissue information that may be displayed to the operator. For example, tissue information that may be displayed to the operator may include a likelihood of tissue voxels being cancerous. While this represents one possible implementation, embodiments of the invention described below may also allow for simultaneous acquisition of transmission and scatter data. Additionally, the system operation steps may be different to conduct scatter measurements on a region of interest (e.g., from measured transmission data or alternate 3D imaging modality) or scatter measurements for a larger region or entire patient body that does not require a prior 3D image to guide the scatter measurement location without departing from the scope of the subject matter described herein.

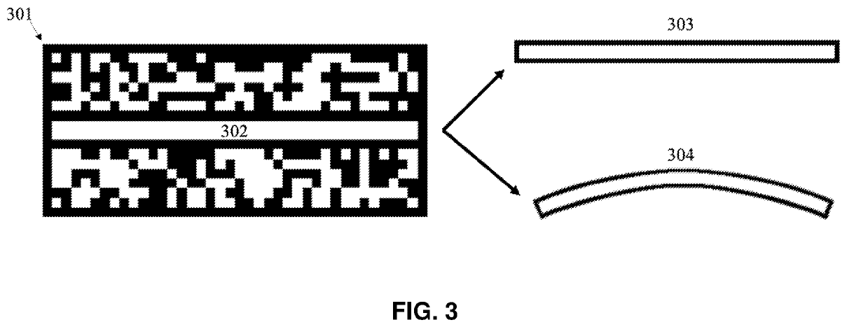

FIG. 3 depicts a schematic of a coded aperture and how the coded aperture can be flat or curved relative to the oncoming scattered X-rays according to an embodiment of the subject matter described herein. One or more coded aperture settings or configurations for optimizing performance may be used. In FIG. 3, a coded aperture 301 is shown, where black regions represent material that absorbs scattered X-rays, while white regions allow X-rays to pass through. This coded aperture shown in FIG. 3 represents one potential pattern (e.g., 2D random), but other potential patterns (e.g., periodic, Fresnel zone plate, random or optimized random, uniformly redundant array) may also be used. Other configurable settings or configurations of the coded apertures include, but are not limited to, for example: (1) feature size, which can affect reconstructed spatial and spectral resolution, and (2) open fraction, which controls the total measured scatter signal strength. A central opening 302 in the coded aperture 301 allows a primary X-ray beam to pass through for transmission measurements without being absorbed or creating undesired background scatter. In one embodiment, the coded aperture 301 may be a flat panel 303 relative to the X-ray source, as shown in FIG. 3. Alternatively, the coded aperture 301 may have a curvature 304 that is beneficial for a larger fan beam and/or the curved X-ray detector arrays used in CT systems. Using a curved coded aperture, the relative angular collimation of features may be maintained for different locations along a fan extent or entry angles of pencil beams. While the coded aperture may include openings that allow the primary beam to pass through, the imaging system may also usc coded apertures with built-in beam blocks or that translate or rotate into or out of position depending on whether transmission or scatter data is being measured.

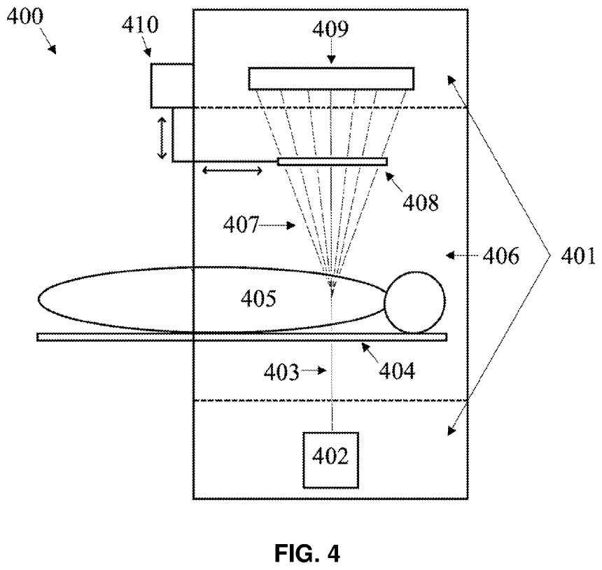

FIG. 4 depicts a side view of a CT system with a motorized mechanism for moving coded aperture into and out of a position to best modulate the detected scattered X-ray signal for scatter or transmission measurements modes, respectively, according to an embodiment of the subject matter described herein. Referring to FIG. 4, the CT system 400 has a gantry enclosure 401, an X-ray source 402, an X-ray primary beam 403, a patient table 404, a patient 405, a CT scanner bore 406, X-ray scatter 407, a coded aperture 408, an X-ray detector 409, and a mechanism 410 for moving the coded aperture 408. Moving the coded aperture out of the beam path is useful for CT transmission measurements, while also allowing for the coded aperture to be moved into a desired position for scatter measurements is useful for optimizing for patient size or relative location of the region of interest within the bore (i.e., proximity of the coded aperture to the patient versus proximity of the coded aperture to the X-ray detector).

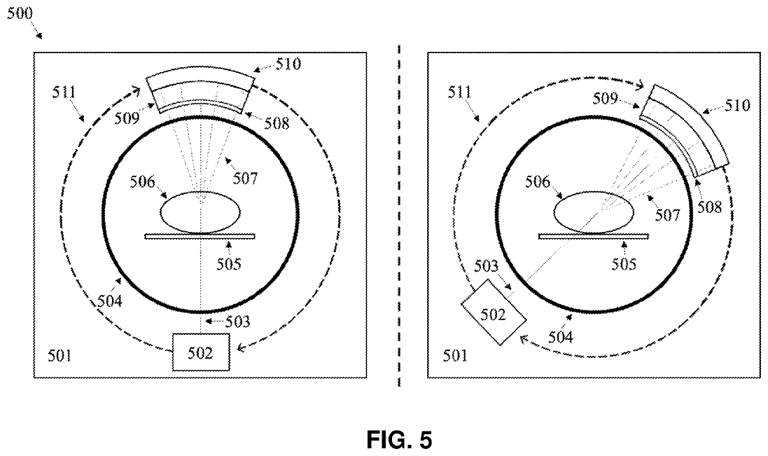

FIG. 5 depicts a schematic of a CT system showing how a primary pencil or fan beam enters the patient from any angle while the coded aperture moves with the X-ray detector for performing measurements according to an embodiment of the subject matter described herein. Whether the coded aperture is positioned or located within the gantry enclosure or inside the CT bore region (moveable or fixed position), the ability for gantry rotation and coded aperture positioning allows for scatter measurements to be conducted at different angles, thereby optimizing the measurement process. Referring to FIG. 5, the imaging system 500 includes a CT gantry enclosure 501, an X-ray source 502, a primary pencil or fan beam 503, CT scanner bore 504, a patient table 505, a patient 506. scattered X-rays 507, a coded aperture 508, a connector between coded aperture and X-ray detector 509, an X-ray detector 510, and a gantry for rotating X-ray components 511. On the left panel of FIG. 5, a scatter measurement is shown that is conducted with the X-ray source 502 at the bottom of the gantry 511, while the right panel shows that scatter measurements may occur at any arbitrary angle relative to the patient 506. This change in measurement angle may be used to optimize for lowering patient dose (resulting from a smaller path through body) or increasing the quality of measurement (by having the target tissue region of interest closer to the exit location of the primary beam from the patient, so less absorption of scattered X-rays occurs). This approach may be used whether the coded aperture is placed within the gantry enclosure or if it is positioned within the borc region.

FIG. 6 depicts a schematic of a CT system showing angled source-side collimation for converting a fan beam to pencil beam for measuring scatter through target regions of a patient according to an embodiment of the subject matter described herein. For implementation of scatter measurements in a manner that may reduce radiation dose, use fewer components, and allow for exploration of regions of interest, FIG. 6 shows one possible implementation. Imaging system 600 includes an X-ray source 601, a primary source pencil beam collimator out of the beam path 602. a primary X-ray fan beam 603, a patient 604, a first region of interest 605 (e.g., suspicious mass), a second region of interest 606, a coded aperture 607, and an X-ray detector 608. In another configuration, as described in greater detail below, the imaging system 600 may further include a collimator 609 creating a pencil beam 610 to measure a first region of interest, the pencil beam 610 targeting the first region of interest, scattered X-rays 611 from the pencil beam 610. In another configuration, as described in greater detail below, the imaging system 600 may further include a collimator 612 at a new angle to create a pencil beam 613 to measure a second region of interest, the pencil beam 613 targeting the second region of interest, and scattered X-rays 614 from the pencil beam 613.

The left panel of FIG. 6 shows how collimation may be out of the primary beam path (or open jaws around the primary beam), which allows for a standard X-ray fan beam to be used for transmission measurements. The coded aperture may be positioned out of the path of the primary X-ray fan beam 603 (or have a slit as shown in FIG. 3) to avoid interacting with the primary beam 603.

The center panel of FIG. 6 shows how collimation may be used to produce a pencil beam that targets a first region of interest, while only generating scatter signals along the pencil beam path. The coded aperture along with post-processing algorithms allows for localization of scatter origin and angle along the pencil beam path, producing momentum transfer spectra of the region of interest that may be used for tissue assessment.

The right panel of FIG. 6 shows how the collimation may be altered to target a second region of interest. For example, the pencil beam angle and relative angle positioning of X-ray components to the patient may be optimized as in FIG. 5 for multiple parameters, including having the pencil beam pass by to the left or right of the X-ray detector while allowing for more of the X-ray detector width to acquire scatter data.

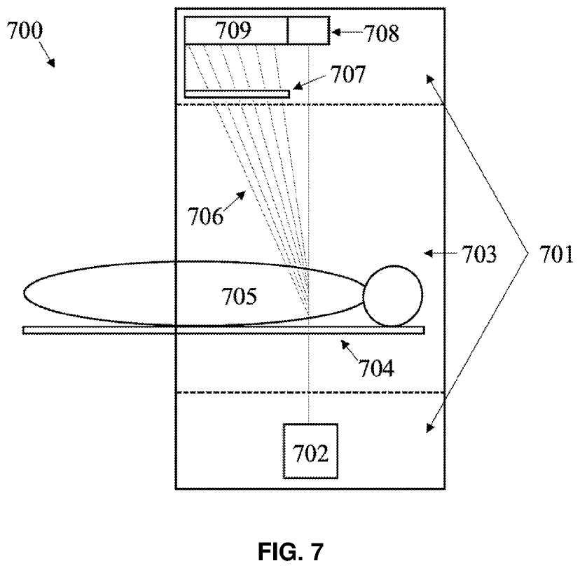

FIG. 7 depicts a schematic of a CT scanner with coded aperture and extra X-ray detector built into a gantry outside of the primary X-ray beam path according to an embodiment of the subject matter described herein. The additional X-ray detector(s), offset from the initial beam path along the bore axis direction, in the system embodiment of the imaging system shown in FIG. 7 allows for simultaneous acquisition of transmission and scatter data or for scatter measurements with minimal shifting of components. Imaging system 700 includes a gantry enclosure 701, an X-ray source 702, a CT scanner bore 703, a patient table 704, a patient 705. scattered X-rays 706, a coded aperture 707, an X-ray transmission detector 708, and an X-ray scatter detector(s) 709. Within FIG. 7, the X-ray detectors for measuring scatter data are positioned outside of the primary beam path along the bore axis direction. Another advantage of this embodiment is that it avoids negative impacts to the scatter data from the primary beam impinging on the X-ray scatter detector. Placement of the X-ray scatter detector 709 and the coded aperture 707 in a different plane relative to the transmission measurement plane (or adjacent but outside of the primary beam path) allows for both data types to be measured simultaneously. These X-ray scatter measurement components may be stationary within the machine or mounted onto the rotating gantry to allow for measurements through patients at different entry angles.

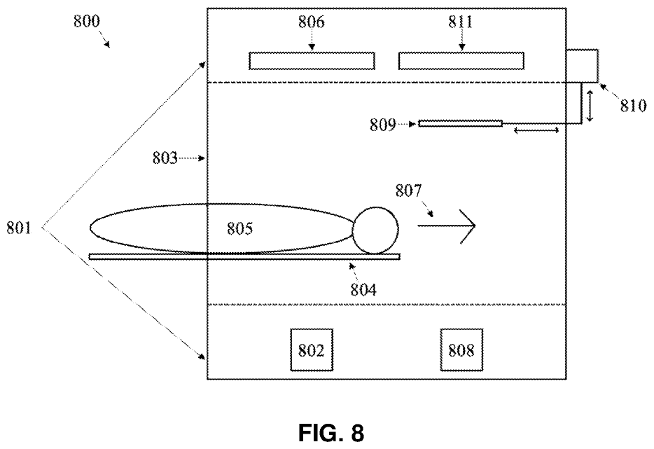

FIG. 8 depicts a schematic of a CT system with separate X-ray sources and X-ray detectors at different locations along the bore axis direction for transmission and scatter measurements according to an embodiment of the subject matter described herein. The separation of transmission and scatter measurement components into different planes relative to patient motion may also be achieved by having larger separation within the gantry enclosure as in FIG. 8. The imaging system 800 includes a gantry enclosure 801, an X-ray source for transmission 802, a CT scanner bore 803, patient table 804, a patient 805, an X-ray transmission detector 806, a patient motion for scatter measurement 807, an X-ray source for scatter 808, a coded aperture 809, a mechanism 810 for moving the coded aperture 809, and an X-ray scatter detector 811. In FIG. 8, the components for each type of measurement exist at different planes of the system along the bore axis direction, which is also the direction of patient motion into the bore. If a different X-ray source and X-ray detector are desired for X-ray scatter measurements, having all components with some separation in the same system may be preferred relative to strategies shown in previous Figures that use the same X-ray source for both transmission and scatter measurements. It may be appreciated that it is possible that the mechanism 810 for moving the coded aperture 809 may not be required if a fixed location for the coded aperture 809 works for all patient sizes/measurements. The inclusion of mechanism 810, however, allows for additional optimizations of the measurement geometry.

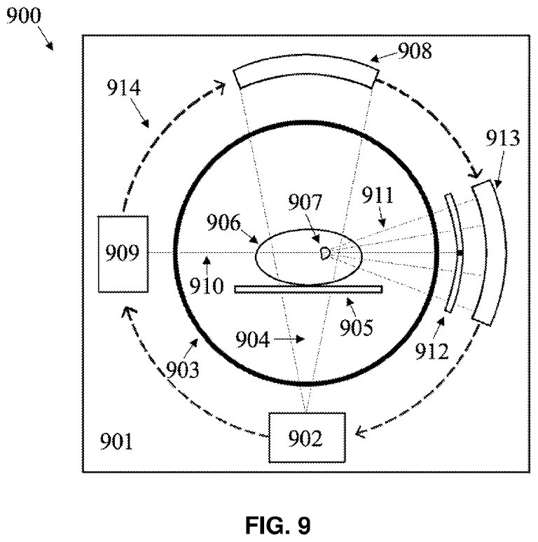

FIG. 9 depicts a schematic of a CT scanner having dual sources and X-ray detectors for transmission and scatter measurements within the same plane according to an embodiment of the subject matter described herein. As an alternative to having separate X-ray components for transmission and scatter measurements at different bore tunnel depths, the components may exist within the same plane but at different angles, as depicted in FIG. 9. Imaging system 900 includes a gantry enclosure 901, an X-ray source for transmission 902, a CT scanner bore 903, an X-ray fan beam for transmission measurements 904, a patient table 905, a patient 906, a region of interest 907 (e.g., suspicious growth), an X-ray transmission detector 908, an X-ray source 909 for scatter, an X-ray pencil beam 910 for scatter measurement, scattered X-rays 911 from pencil beam, a coded aperture 912 with a central beam block for pencil beam, an X-ray scatter detector, and a gantry 914 for rotating X-ray components. This approach is compatible with CT scanners that have perpendicular dual sources. While the system of FIG. 9 depicts the second source and X-ray detector being dedicated for scatter measurements, both source and X-ray detector pairs may be used for transmission and scatter measurements, allowing for faster measurement speeds of both data types, without departing from the scope of the subject matter described herein. The X-ray detector arrays shown in FIG. 9 may allow for both sets of X-ray sources and X-ray detectors to simultaneously acquire transmission and scatter data. Alternatively, methods for changing between transmission and scatter measurement modes previously discussed may be used for one or both X-ray source and X-ray detector pairs, allowing for a transmission measurement to be acquired first, followed by switching to a scatter measurement mode for one or both sources.

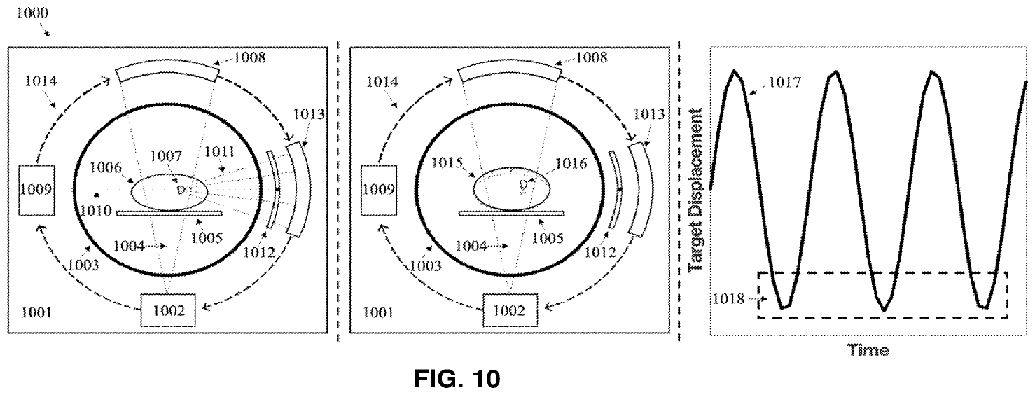

The described imaging system may be used for analyzing tissue in a variety of locations within a patient. For tissue or suspicious growths within regions like the brain, there is minimal motion that is anticipated during measurement. In contrast, if measuring scatter properties of tissue in the lungs, patient breathing can cause tissue motion during measurements. During standard CT imaging, patients arc asked to hold their breath to reduce the impact of this type of motion. For scatter measurements, while the system may potentially acquire all data needed during a single breath hold, it is possible that certain embodiments of the method, such as those that include whole body or whole slice scatter measurements, would take longer. To account for this, the method shown in FIG. 10 may be implemented.

FIG. 10 depicts a schematic illustrating accounting for patient motion during scatter measurements according to an embodiment of the subject matter described herein. Imaging system 1000 includes a gantry enclosure 1001, an X-ray source for transmission 1002, a CT scanner bore 1003, an X-ray fan beam 1004, a patient table 1005, a patient 1006, a region of interest 1007 (e.g., suspicious growth), an X-ray transmission detector 1008, an X-ray source 1009 for scatter, an X-ray pencil beam 1010, scattered X-rays 1011 from pencil beam 1010, a coded aperture plus beam block 1012, an X-ray scatter detector 1013, and a gantry 1014 for rotating X-ray components. In another embodiment, discussed in greater detail below, the imaging system 1000 may also include a patient 1015 breathing causing expansion/motion of the body and a region of interest 1016 moved to new location due to patient motion. According to another aspect, a plot of patient motion is shown where the patient motion is measured by transmission or other motion tracking techniques. For example, fiducial markers on patients and cameras may show target mass moving up and down over time from breathing 1017. A time window 1018 in displacement is shown when the patient has exhaled and the region of interest is in a desired location for scatter measurement. The frequency 1017 of target mass moving up and down over time may be used to pulse on and off the X-ray beam (or open and close an X-ray source window) being used for scatter measurements so that scatter data is obtained from a consistent patient positioning, if this is required due to scan times longer than a reasonable patient breath hold for regions where patient motion cannot be prevented.

The left panel of FIG. 10 shows how transmission and scatter measurements can be acquired on a patient. The central panel of FIG. 10 shows how scatter measurement may be gated due to patient motion. The right panel of FIG. 10 shows how patient motion may be tracked. Using this type of gating or motion management, scatter measurements of moving targets may produce more accurate reconstructions because less or none of the data collection occurs when the region of interest is not within the primary beam path or is changing location within this path.

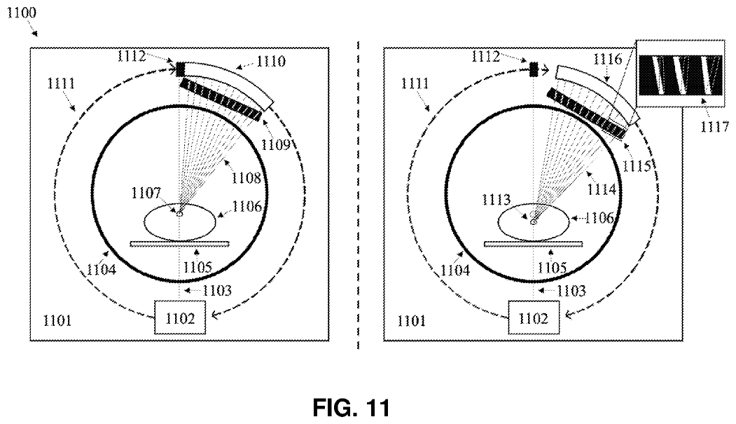

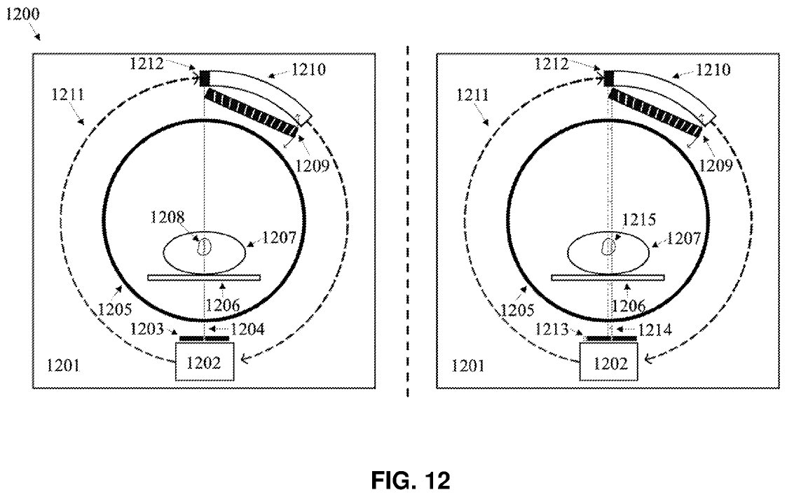

FIG. 11 and FIG. 12 relate to an embodiment of the present invention for measuring specific tissue regions within the body. The measured tissue regions may be user-defined. While the coded aperture has previously been shown at some distance between the X-ray detector and irradiated tissue (and in FIG. 3 it is shown with a 2D random pattern), the coded aperture may also be placed immediately adjacent to the patient or X-ray detector. If the coded aperture is a thick, periodic pattern (e.g., sinusoidal) against the X-ray detector with angled openings targeting a specific point within the body, a primary pencil beam may be used to allow for scatter measurement of an individual voxel within the patient. This embodiment of the coded aperture may be a single attenuating component with openings or a set of attenuating components arranged in a pattern, that together make up an effective coded aperture for modulating the measured scatter. The thick openings in either arrangement may be controlled to focus the aperture at a specific point in the imaging system, particularly within the body of the patient to focus on the scatter from a particular point.

FIG. 11 depicts a schematic of a CT system using a highly focused coded aperture near the X-ray detector for scatter measurement of single point along a pencil beam path according to an embodiment of the subject matter described herein. Referring to FIG. 11, imaging system 1100 includes a gantry enclosure 1101, an X-ray source 1102, a X-ray pencil beam 1103, a CT scanner bore 1104, a patient table 1105, patient 1106, a first scatter measurement spot 1107. scattered X-rays 1108 from the first measurement spot, a highly focused coded aperture collimator 1109 accepting scattered X-rays from the first spot 1107, an X-ray detector 1110, a gantry 1111 for rotating X-ray components, a beam block 1112 for primary X-ray beam to reduce background scatter, a second scatter measurement spot 1113, scattered X-rays 1114 from the second measurement spot 1113, moved focused coded aperture 1115 for measuring scattered X-rays from the second measurement spot, a X-ray detector 1116 that can also move independent of X-ray source if needed for desired range of scatter angle measurements, and a zoomed in window 1117 showing how the highly focused coded aperture collimation may be tilted by rotation of the overall component or motorized motion of individual pieces making up the coded aperture to change the focal point location along the X-ray pencil beam. In contrast to the embodiments described above where a coded aperture and X-ray detector system are used for simultaneous scatter measurement of many tissue regions illuminated by a pencil or fan beam, the embodiment shown in FIG. 11 may be used if the reduction of this multiplexed measurement to a measurement approach of a single point presents as optimal for a given task.

FIG. 12 depicts a schematic of raster scanning of the pencil beam and highly focused coded aperture shown in FIG. 11 to measure multiple voxels according to an embodiment of the subject matter described herein. This implementation measures scatter data for individual points and may be used for planar or volumetric imaging, by raster scanning the pencil beam and focal point of highly focused coded aperture over different locations within a patient, as in FIG. 12. Imaging system 1200 includes a gantry enclosure 1201, an X-ray source 1202, an X-ray pencil beam collimation that can move 1203, an X-ray pencil beam 1204, a CT scanner bore 1205, a patient table 1206, a patient 1207, a first column of scatter measurement spots 1208, a highly focused coded aperture that can tilt to change focal point location 1209, an X-ray detector with potential independent motion relative to X-ray source 1210, a gantry for rotating X-ray components 1211, a beam block for primary X-rays 1212, an X-ray pencil beam collimator that has been shifted to measure new column of spots 1213, a shifted X-ray pencil beam 1214, and a second column of scatter measurement spots 1215. If scatter measurements occur rapidly, the measurement described by FIG. 11 and FIG. 12 may rapidly measure areas through fast raster scanning.

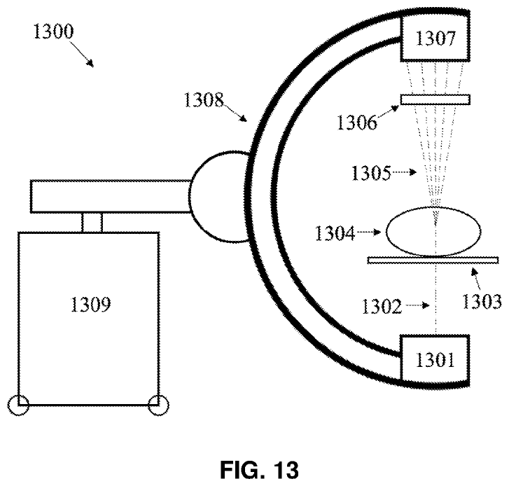

While several embodiments of the in vivo scatter imaging system have been described as combinable with a standard transmission-based CT scanner, there are other X-ray imaging architectures that the invention could be combined with. FIG. 13 presents an embodiment where in vivo coded aperture scatter imaging is combined with a C-arm transmission-X-ray system. Given that C-arm systems can be used during surgical procedures, combining this invention within the C-arm architecture is of value for imaging applications that do not utilize devices in the form of CT scanners. Shown in FIG. 13, is the imaging system 1300 including an X-ray source 1301, primary X-ray beam 1302, patient table 1303, patient 1304, X-ray scatter 1305, coded aperture 1306, X-ray detector 1307, C-arm 1308, and C-arm body 1309. As described in previous Figures, the coded aperture could be connected to the C-arm and positioned to an ideal location dependent on patient size and application.

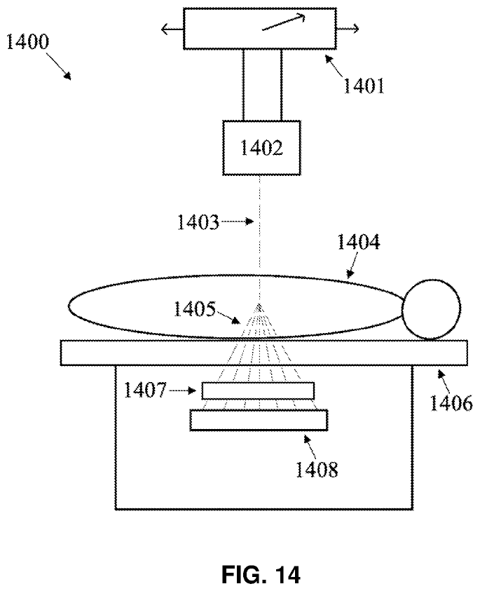

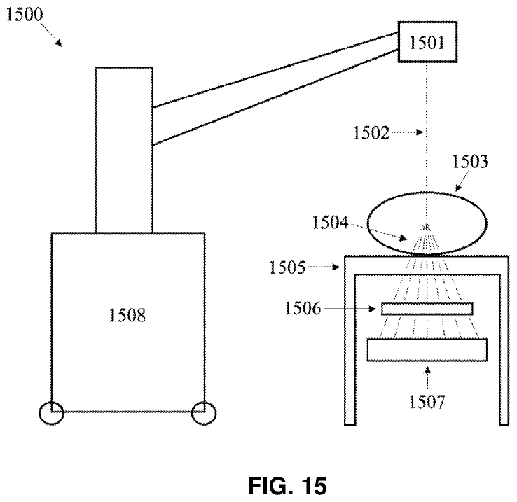

An additional embodiment of the invention with an alternative X-ray transmission imaging system is shown in FIG. 14, where the in vivo coded aperture scatter imaging technology is combined with a ceiling-mounted X-ray transmission system. The ceiling-mounted X-ray transmission system is often used for 2D X-ray imaging applications, and it is beneficial during these exams to collect scatter data for additional tissue contrast. Within FIG. 14, the imaging system 1400 is composed of the moveable ceiling mount 1401, an X-ray source 1402, a primary X-ray beam 1403, a patient 1404, X-ray scatter 1405, patient table 1406, coded aperture 1407, and X-ray detector 1408. Another embodiment of the invention, similar to that of the ceiling-mounted X-ray system of FIG. 14, is the combination of in vivo coded aperture X-ray scatter imaging within a portable X-ray imaging system as shown in FIG. 15. X-ray imaging systems that are portable are used within clinical settings when it is advantageous to bring the imaging device to a patient without the need to transport them to a separate imaging room. In these applications, an X-ray detector can be placed behind the patient/within their bed for data acquisition. In the same way, a coded aperture may be positioned behind the patient but before the X-ray detector for spatial encoding of X-ray scatter signal. In FIG. 15, the imaging system 1500 contains an X-ray source 1501, primary X-ray beam 1502, patient 1503, X-ray scatter 1504, patient table 1505, coded aperture 1506, and X-ray detector 1507. While the embodiments of in vivo coded aperture X-ray scatter imaging in FIGS. 13-15 are provided as additional examples for accompanying technologies the invention can be combined with, this list is not meant to be exhaustive.

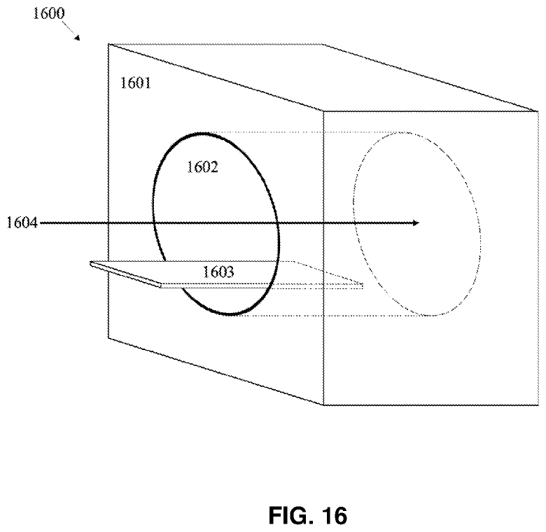

FIG. 16 depicts a schematic of a general CT system with an arrow passing through the bore of the gantry to indicate the axis that is referred to as the bore axis or patient motion axis according to an embodiment of the subject matter described herein. For visualization, FIG. 16 depicts a general CT scanner with the imaging system 1600 including the primary CT scanner frame/gantry 1601, the CT bore 1602, the patient table 1603, and the bore axis arrow 1604. This schematic allows for case of understanding what is referred to herein as the bore axis or patient motion axis, to which specific embodiments of the subject matter refer.

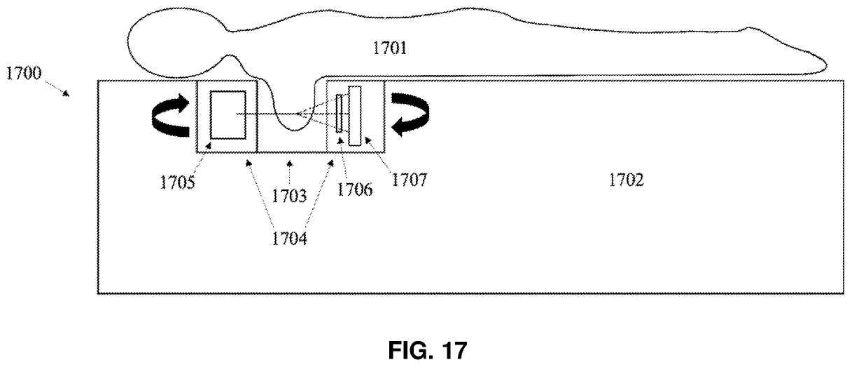

FIG. 17 depicts a schematic of a CT bore and gantry system with a coded aperture built into a table for the purpose of receiving and scatter imaging the breast of a patient during breast CT imaging according to an embodiment of the subject matter described herein. In embodiments of the present invention designed for specific clinical tasks, such as imaging specific regions of the human body (e.g. head, breast), a coded aperture is positioned within the bore and gantry system to enable scatter imaging. For demonstration of one such implementation, FIG. 17 depicts a breast CT system with a coded aperture for scatter imaging. Within FIG. 17 the schematic of a breast CT imaging system 1700 includes a patient 1701 positioned prone on a table 1702, where the table has a vertical bore 1703 surrounded by a vertically oriented gantry 1704 that is capable of rotating around the patient's breast positioned within the bore. Furthermore, the gantry components for rotation around the breast include an X-ray source 1705 that can include collimation for shaping or aiming the primary beam, a coded aperture 1706 for modulating the scattered X-rays, and an X-ray detector 1707 for receiving the transmitted and scattered signals. In this form, a mammogram screening or diagnostic exam can be conducted without breast compression, potentially using X-ray cone or fan beam geometry. If a suspicious mass is identified, scatter imaging, with any X-ray beam geometry using stationary or rotating components, could be conducted to obtain further diagnostic information. Additionally, the system could be designed with a fan beam geometry and coded aperture with a slit for the primary beam to pass through, or with the coded aperture positioned out of the primary beam path, allowing for simultaneous 3D transmission and scatter imaging, producing a 4D image that could be utilized for breast screening or diagnostic applications. This breast imaging CT example is illustrative of one embodiment of the present invention, whereas other embodiments include head CT imaging systems or other specialized CT systems for imaging specific body regions.

In highlighting additional embodiments of collimators mentioned within, the following paragraph provides as example and not limitation what can be understood as a collimator. X-ray collimators can be multi-stage, meaning there are elements shaping or collimating the X-ray beam at different distances from the X-ray source. Many collimators on modern imaging systems have two stages of collimation, with two pairs of jaws closest to the X-ray source for creating square or rectangle illumination, with another two pairs of jaws further away from the X-ray source to further control divergence and absorb some of the scattered X-rays generated by the first pair of jaws. Additionally, there can be any arbitrary number of collimation stages, for example, one embodiment of the disclosed invention can have a third stage within the primary collimator or closer to the patient, that can serve as a guard collimator that does not interact with or absorb the primary X-ray beam but is only meant to absorb scattered X-rays created by the primary beam interacting with the prior collimator stages. Commonly used in radiation therapy, multileaf collimators allow for more complex primary X-ray beam shapes beyond rectangles and could be utilized within an embodiment of the disclosed invention. In a more simplified case beyond jaws and multileaf collimators, a collimator could have a pin hole collimator that could be moved into the beam path to change the X-ray primary beam shape from cone or fan to become a pencil beam X-ray geometry. The collimator further can be constructed not as individual stages, but as one solid manufactured part (whether cut from a bulk material or created by additive methods) whose position and orientation could be controlled for imaging desired regions within a patient. Any of the described versions of the collimator can be used individually or in combination with each other and are provided as examples not meant to limit given that there are many known collimation techniques that could be implemented.

Beyond variations in collimator choices, there are a variety of methods that can be utilized for the creation of a coded aperture or encoder for assisting in localizing scattered X-ray origins. As examples, not intended to be limiting, the coded aperture can be CNC cut from a sheet of metal, created by waterjet or laser cutting, created by additive manufacturing methods including 3D printing, or photochemically etched on metal sheets. While these different methods have various limitations and benefits, the resulting manufactured parts would be coded aperture as described within the disclosed embodiments of the invention, useful for a range of imaging applications including telescopes and camera systems, but here applied to X-ray scatter imaging.

On the topic of variations that can be selected in design elements of the disclosed invention while still being within the scope of the disclosed invention, it is possible that an embodiment of the disclosed invention could utilize alternatives to physics forward modeling for the estimation of tissue properties, while still utilizing a coded aperture within the imaging system. There is the potential for operating classification algorithms, both rules-based and machine-learned on the raw encoded scatter data that is detected, without reconstructing explicitly scattered X-ray spectra within pixels/voxels/toxels of the image. Additionally, it is possible that other reconstruction methods could utilize modeling not typically defined as forward modeling, including inverse modeling or machine-learned modeling to obtain the desired tissue property data from the encoded scattered X-ray measurement. For our discussion of the disclosed invention, we utilize the common term forward model to describe what is utilized when accounting for the physics of this computational system, but we do not intend for this to be limiting in embodiments that use alternate forms of modeling or no modeling at all for estimating tissue properties from measured encoded X-ray scatter data.

For reconstruction algorithms that can be utilized in an embodiment of the disclosed invention for utilizing measured encoded scatter data to reconstruct data useful for tissue property measurements, there are a range of reconstruction algorithms that can be utilized that would not depart from the spirit of the disclosed invention. Reconstruction algorithms can be analytical (e.g., back-projection algorithms utilized in CT imaging), iterative (e.g., maximum likelihood estimation), machine-learned, or any combination of these reconstruction types, to provide examples that are not meant to be limiting. The use of common reconstruction algorithms, advanced combinations of reconstruction techniques, or new reconstruction algorithms yet to be invented for coded aperture scatter imaging reconstruction are within the scope of the disclosed invention.

In one embodiment, an in vivo tissue imaging system is disclosed. The imaging system comprises an X-ray source for irradiating in vivo tissue with an initial X-ray beam. The imaging system further comprises a collimator positioned between the X-ray source and the tissue to direct the initial X-ray beam. The imaging system further comprises an X-ray detector array comprising an arrangement of X-ray detecting elements in at least two dimensions, where a plurality of the X-ray detecting elements are positioned to detect scattered X-ray radiation from the initial X-ray beam passing through the tissue. The imaging system further comprises a coded aperture positioned between the tissue and the X-ray detector array to modulate the scattered X-ray radiation from the tissue detected by the X-ray detector array. The imaging system further comprises a control system that comprises a processor and memory. The processor is configured to configure the imaging system for an X-ray scatter measurement based on configuration data, where the data includes at least one of: timing data, location data, and orientation data for the X-ray source and a plurality of the X-ray detecting elements. The processor is further configured to enact the X-ray scatter measurement with the configured imaging system. The processor is further configured to receive data representing the detected scattered X-ray radiation from the X-ray detector array. The processor is further configured to analyze the received data to generate a representation of the irradiated tissue. The representation is generated from the received X-ray scatter data based on the configuration data. The representation comprises a spatially resolved property of the irradiated tissue.

In an embodiment of the imaging system described above, the representation of the irradiated tissue includes indications of potentially cancerous regions within the irradiated tissue.

In an embodiment of the imaging system described above, the processor is further configured for reconstructing an estimate of spatially resolved X-ray scatter spectra of the irradiated tissue using the received X-ray scatter data and a forward model of the imaging system. In the embodiment, the estimate of spatially resolved X-ray scatter spectra of the irradiated tissue is used to generate the representation of the irradiated tissue.

In an embodiment of the imaging system described above, the processor is further configured for selecting an existing forward model of the imaging system using the configuration data or generating a new forward model of the imaging system using the configuration data.

In an embodiment of the imaging system described above, computing a spatially resolved estimate of the momentum transfer spectra of the irradiated tissue from the X-ray scatter data. In the embodiment, the processor is further configured for computing a spatially resolved estimate of a tissue property of the irradiated tissue using the spatially resolved estimate of the momentum transfer spectra of the irradiated tissue.

In an embodiment of the imaging system described above, the processor is further configured for using a reference library of existing tissue momentum transfer spectra in combination with the spatially resolved estimate of the momentum transfer spectra of the irradiated tissue to compute the spatially resolved estimate of a tissue property of the irradiated tissue.

In an embodiment of the imaging system described above, the processor is further configured for using a classification algorithm to compute the spatially resolved estimate of a tissue property of the irradiated tissue.

In an embodiment of the imaging system described above, the processor is further configured for using a machine learning algorithm for classification in the computation of the spatially resolved estimate of a tissue property.

In an embodiment of the imaging system described above, the processor is further configured for using a rules-based algorithm for classification in the computation of the spatially resolved estimate of a tissue property.

In an embodiment of the imaging system described above, at least one of the coded aperture, the X-ray source, the collimator, or a plurality of the X-ray detecting elements may be moveable. In the embodiment, the processor is further configured for receiving configuration data comprising at least one of location data or orientation data for the coded aperture, X-ray source, collimator, or plurality of X-ray detecting elements.

In an embodiment of the imaging system described above, the X-ray source is an X-ray generator, controllable by the processor. In the embodiment, the configuration data further comprises at least one of X-ray source current or X-ray source voltage.

In an embodiment of the imaging system described above, the collimator includes a controllable opening, controllable by the processor. In the embodiment, the configuration data further comprises at least one of timing data for the controllable opening, size of the collimator opening to configure spatial extent of the initial X-ray beam or shape of the collimator opening to configure cross-sectional shape of the initial X-ray beam.

An embodiment of the imaging system described above further comprises a moveable filter, controllable by the processor, positioned between the X-ray source and the tissue when the X-ray source is irradiating the tissue to modify the energy spectrum and irradiance of the initial X-ray beam. In the embodiment, the configuration data further comprises at least one of timing data, location data, or orientation data for the filter.

In an embodiment of the imaging system described above, the tissue to be irradiated is moveable and controllable by the processor. In the embodiment, the configuration data further comprises at least one of timing data, location data, or orientation data for the tissue.

In an embodiment of the imaging system described above, at least one of the coded aperture, the X-ray source, the collimator, or a plurality of the X-ray detecting elements is moveable and controllable by the processor. In the embodiment, the configuration data further comprises at least one of timing data, location data, or orientation data for the at least one moveable X-ray source, collimator, coded aperture, or plurality of X-ray detecting elements.

In an embodiment of the imaging system described above, at least one of the X-ray source or a plurality of the X-ray detecting elements is moveable and controllable by the processor. In the embodiment, the configuration data further comprises at least one of timing data, location data, or orientation data for the at least one moveable X-ray source or plurality of X-ray detecting elements. In the embodiment, at least one moveable X-ray source or plurality of X-ray detecting elements is operable while in motion.

In an embodiment of the imaging system described above, the at least one moveable X-ray source, collimator, coded aperture, or plurality of X-ray detecting elements is configurable to enable a plurality of different perspectives of the tissue for an X-ray scatter measurement. In the embodiment, the processor is further configured for analyzing received X-ray scatter data from a plurality of different perspectives of the tissue to generate the representation of the irradiated tissue.

In an embodiment of the imaging system described above, the at least one moveable X-ray source, collimator, coded aperture, or plurality of X-ray detecting elements is rotatable about the tissue.

In an embodiment of the imaging system described above, the processor is further configured for computing an estimate of a data quality metric for the resulting X-ray scatter data from the configuration data. In the embodiment, the processor is further configured for computing optimized configuration data using the estimated X-ray scatter data quality metric. In the embodiment, the optimized configuration data is computed to define a perspective for an X-ray scatter measurement of a point in the tissue to satisfy at least one of minimizing the attenuation of the initial X-ray beam along the initial X-ray beam path to the point in the tissue or minimizing the attenuation of the scattered X-ray signal from the point in the tissue to an X-ray detecting element.

In an embodiment of the imaging system described above, the processor is further configured for computing an estimate of radiation dose to the patient for an X-ray scatter measurement from the configuration data. In the embodiment, the processor is further configured for computing an estimate of a data quality metric for the resulting X-ray scatter data from the configuration data. In the embodiment, the processor is further configured for computing optimized configuration data using the estimated radiation dose and the estimated X-ray scatter data quality metric. In the embodiment, the processor is further configured for configuring the imaging system for an X-ray scatter measurement based on the computed configuration data.

An embodiment of the imaging system described above further comprises a camera for recording the patient. In the embodiment, the processor is further configured for receiving recording data from the camera. In the embodiment, the processor is further configured for analyzing the recording data from the camera to compute an estimate of patient motion. In the embodiment, the processor is further configured for using the computed estimate of patient motion in combination with received X-ray scatter data to generate a representation of the irradiated tissue that accounts for an effect of patient motion.

In an embodiment of the imaging system described above, at least one of the X-ray source, the collimator, the coded aperture, or a plurality of the X-ray detecting elements may be further configurable by the processor. In the embodiment, the processor is further configured for analyzing the estimate of patient motion to compute configuration data for the at least one further configurable X-ray source, collimator, coded aperture, or plurality of X-ray detecting elements. In the embodiment, the configuration data is computed to minimize an effect of patient motion on the X-ray scatter data. In the embodiment, the processor is further configured for configuring the imaging system for an X-ray scatter measurement based on the computed configuration data.

An embodiment of the imaging system described above a beam block disposed in the path of the initial X-ray beam between the tissue and the X-ray detector array to block the initial X-ray beam during an X-ray scatter measurement.

In an embodiment of the imaging system described above, the coded aperture includes a periodic pattern in at least one dimension having a single attenuating component with openings focused at a millimeter-scale focal point 100-2000 millimeters from the coded aperture 100-2000. In the embodiment, the coded aperture is moveable and controllable by the processor. In the embodiment, the tissue to be irradiated is moveable and controllable by the processor. In the embodiment, the configuration data further comprises at least one of timing data for the tissue, location data for the tissue, orientation data for the tissue, timing data for the coded aperture, location data for the coded aperture, or orientation data for the coded aperture to direct the focal point of the coded aperture at a point in the tissue.

In an embodiment of the imaging system described above, the coded aperture comprises a set of moveable and controllable attenuating components arranged in a periodic pattern in at least one dimension. In the embodiment, the tissue to be irradiated is moveable and controllable by the processor. In the embodiment, the configuration data further comprises at least one of timing data for the tissue, location data for the tissue, orientation data for the tissue, timing data for the attenuating components, location data for the attenuating components, or orientation data for the attenuating components to focus the coded aperture at a millimeter-scale focal point 100-2000 millimeters from the coded aperture and direct the focal point at a point in the tissue.

In an embodiment of the imaging system described above, the processor is further configured for transmitting the representation of the irradiated tissue to a display.

An embodiment of the imaging system described above further comprises at least one of a secondary collimator positioned between the tissue and the X-ray detector array to collimate the scattered X-ray radiation or a filter positioned between the tissue and the X-ray detector array to modify the energy spectrum and irradiance of the scattered X-ray radiation.

An embodiment of the imaging system described above further comprises a secondary coded aperture positioned between the X-ray source and the tissue configured for modulating the initial X-ray beam.

In an embodiment of the imaging system described above, a plurality of the X-ray detecting elements are positioned to detect X-rays transmitted directly through the tissue from the initial X-ray beam such that the in vivo imaging system operates as an X-ray transmission imaging system. In the embodiment, the processor is further configured for configuring the imaging system for an X-ray transmission measurement based on the configuration data. In the embodiment, the configuration data further comprises at least one of timing data, location data, or orientation data for the X-ray source and a plurality of the X-ray detecting elements. In the embodiment, the processor is further configured for performing the X-ray transmission measurement with the configured imaging system. In the embodiment, the processor is further configured for receiving data representing the detected transmitted X-ray radiation from the X-ray detector array. In the embodiment, the processor is further configured for computing X-ray radiodensity tissue images from the received X-ray transmission data.

In an embodiment of the imaging system described above, the configuration data for an X-ray transmission measurement is different from the configuration data for an X-ray scatter measurement.

In an embodiment of the imaging system described above, the coded aperture is moveable and controllable by the processor. In the embodiment, the coded aperture is moveable to a position in which the coded aperture is positioned between the tissue and the X-ray detector array to modulate the scattered X-ray signal during an X-ray scatter measurement. In the embodiment, the configuration data further comprises at least one of timing data, location data, or orientation data for the coded aperture.

In an embodiment of the imaging system described above, at least one of the X-ray source, the collimator, or a plurality of the X-ray detecting elements is moveable and controllable by the processor. In the embodiment, the configuration data further comprises at least one of timing data, location data, or orientation data for the at least one moveable X-ray source, collimator, or plurality of X-ray detecting elements such that a plurality of the X-ray detecting elements is offset from the initial X-ray beam path for an X-ray scatter measurement to increase the relative solid angular coverage of the X-ray detector array for a range of X-ray scatter angles from a point in the tissue.

In an embodiment of the imaging system described above, at least one of the X-ray source, the collimator, the coded aperture, or a plurality of the X-ray detecting elements is further configurable by the processor. In the embodiment, the configuration data specifies a configuration for an X-ray scatter measurement subsequent to an X-ray transmission measurement such that the tissue volume imaged during the subsequent X-ray scatter measurement is a subregion of the tissue volume imaged during the X-ray transmission measurement.

In an embodiment of the imaging system described above, the processor is further configured for receiving user input for selecting a subregion of tissue for the X-ray scatter measurement. In the embodiment, the processor is further configured for computing the configuration data for the at least one further configurable X-ray source, collimator, coded aperture, or plurality of X-ray detecting elements using the user input. In the embodiment, the processor is further configured for configuring the imaging system for the X-ray scatter measurement based on the computed configuration data.

In an embodiment of the imaging system described above, the processor is further configured for transmitting the X-ray radiodensity tissue image to a display. In the embodiment, the user input comprises an indication of a subregion of the displayed X-ray radiodensity tissue image.

In an embodiment of the imaging system described above, the processor is further configured for computing a spatially resolved estimate of the likelihood of cancer from the X-ray radiodensity tissue image. In the embodiment, the processor is further configured for computing regions of interest in the X-ray radiodensity tissue image using the spatially resolved estimate of the likelihood of cancer. In the embodiment, the processor is further configured for transmitting the region of interest data to the display.

In an embodiment of the imaging system described above, the processor is further configured for using machine learning algorithms to compute the regions of interest in the X-ray radiodensity tissue image.

An embodiment of the imaging system described above further comprises a beam block to block the initial X-ray beam during an X-ray scatter measurement. In the embodiment, the beam block is moveable and controllable by the processor. In the embodiment, the beam block is moveable to a position in which the beam block is positioned in the path of the initial X-ray beam between the tissue and the X-ray detector array to block the initial X-ray beam during an X-ray scatter measurement. In the embodiment, the configuration data further comprises at least one of timing data, location data, or orientation data for the beam block.

In an embodiment of the imaging system described above, the processor is further configured for configuring the imaging system for an X-ray scatter measurement and an X-ray transmission measurement that occur synchronously.

In an embodiment of the imaging system described above, the processor is further configured for overlaying the representation of the tissue generated from the X-ray scatter data with the X-ray radiodensity tissue image computed from the X-ray transmission data. In the embodiment, the processor is further configured for transmitting the overlaid transmitting the overlaid representation and image to a display.

In an embodiment of the imaging system described above, the processor is further configured for the processor is further configured for analyzing the received X-ray transmission data in combination with the received X-ray scatter data to generate the representation of the irradiated tissue.

In an embodiment of the imaging system described above the X-ray source includes multiple X-ray sources. In the embodiment, at least one X-ray source is configured for X-ray transmission measurements and at least one X-ray source is configured for X-ray scatter measurements.

In an embodiment of the imaging system described above, at least one of the X-ray detecting elements is configured to detect transmitted X-ray radiation during the X-ray transmission measurement and to detect scattered X-ray radiation during the X-ray scatter measurement.

In an embodiment of the imaging system described above, the processor is further configured for analyzing the received X-ray transmission data to compute an estimate of patient motion. In the embodiment, the processor is further configured for using the estimate of patient motion in combination with the received X-ray scatter data to generate a representation of the irradiated tissue that accounts for an effect of patient motion.

In an embodiment of the imaging system described above, at least one of the X-ray source, the collimator, the coded aperture, or a plurality of the X-ray detecting elements is further configurable by the processor. In the embodiment, the processor is further configured for analyzing the received X-ray transmission data to compute configuration data for the at least one further configurable X-ray source, collimator, coded aperture, or plurality of X-ray detecting elements. In the embodiment, the configuration data is computed to minimize an effect of patient motion on the X-ray scatter data. In the embodiment, the processor is further configured for configuring the imaging system for an X-ray scatter measurement based on the computed configuration data.

In an embodiment of the imaging system described above, at least one of the X-ray source, the collimator, or a plurality of the X-ray detecting elements is moveable and controllable by the processor. In the embodiment, the configuration data further comprises at least one of timing data, location data, or orientation data for the at least one moveable X-ray source, collimator, or plurality of X-ray detecting elements to enable a plurality of different perspectives of the tissue for an X-ray transmission measurement. In the embodiment, the processor is further configured for computing X-ray transmission computed tomography (CT) reconstructions from the received X-ray transmission data from a plurality of perspectives of the tissue, such that the imaging system operates as an X-ray transmission computed tomography (CT) imaging system.

In an embodiment of the imaging system described above, at least one of the X-ray source or a plurality of the X-ray detecting elements is rotatable about the tissue and controllable by the processor. In the embodiment, the configuration data further comprises at least one of timing data, location data, or orientation data for the at least one rotatable X-ray source or plurality of X-ray detecting elements to enable a plurality of different perspectives of the tissue for an X-ray transmission measurement.

An embodiment of the imaging system described above further comprises a bore in which the tissue is located, a rotatable gantry around the bore, and an enclosure around the gantry. In the embodiment, the X-ray source and a plurality of the X-ray detecting elements are rotatable about the tissue and controllable by the processor. In the embodiment, the configuration data further comprises at least one of timing data, location data, or orientation data for the rotatable X-ray source and plurality of X-ray detecting elements to enable a plurality of different perspectives of the tissue for an X-ray transmission measurement. In the embodiment, the rotatable X-ray source and plurality of X-ray detecting elements are attached to and rotate with the gantry.

In an embodiment of the imaging system described above, the coded aperture is within the gantry enclosure of the X-ray transmission computed tomography (CT) imaging system.

In an embodiment of the imaging system described above, the coded aperture is within the bore of the X-ray transmission computed tomography (CT) imaging system.

In an embodiment of the imaging system described above, the coded aperture and the plurality of X-ray detecting elements positioned to detect the scattered X-ray signal are maintained at a location along the bore axis direction in the X-ray transmission computed tomography (CT) imaging system such that the initial X-ray beam does not impinge on the coded aperture or the plurality of X-ray detecting elements positioned to detect the scattered X-ray signal.

In an embodiment of the imaging system described above, the X-ray source includes multiple X-ray sources, with the X-ray sources positioned within the imaging system at different locations along the bore or longitudinal axis direction. In the embodiment, at least one X-ray source is configured for X-ray transmission measurements and at least one X-ray source is configured for X-ray scatter measurements.

In an embodiment of the imaging system described above, the X-ray source includes multiple X-ray sources, with the X-ray sources positioned within the imaging system at the same location along the bore or longitudinal axis direction. In the embodiment, at least one X-ray source is configured for X-ray transmission measurements and at least one X-ray source is configured for X-ray scatter measurements.

In an embodiment of the imaging system described above, the gantry is translatable along the bore axis direction. In the embodiment, the rotatable X-ray source and plurality of X-ray detecting elements attached to the gantry are translatable and controllable by the processor. In the embodiment, the translatable and rotatable X-ray source and plurality of X-ray detecting elements attached to the gantry are operable while in motion. In the embodiment, the processor is further configured for configuring the rotatable and translatable X-ray source and plurality of X-ray detecting elements to be in motion during the X-ray transmission measurement while the gantry is simultaneously rotating about the bore axis direction and translating along the bore axis direction.

An embodiment of the in vivo tissue imaging system described above is integrated into a C-arm X-ray imaging system, a ceiling mounted X-ray imaging system, or a portable/mobile X-ray imaging system.

In an embodiment of the imaging system described above, a plurality of the X-ray detecting elements are positioned to detect X-rays transmitted directly through the tissue from the primary X-ray beam such that the imaging system operates as an X-ray transmission imaging system, and the processor is further configured for configuring the imaging system for performing an X-ray transmission measurement based on the configuration data. The configuration data further comprises the orientation of the primary beam relative to the bore axis and the exposure time for the X-ray transmission measurement. The processor is further configured for performing the X-ray transmission measurement with the configured imaging system. The processor is further configured for receiving data representing transmitted X-ray radiation detected by the X-ray detector array. The processor is further configured for computing an X-ray radiodensity tissue image from the received X-ray transmission data.

In an embodiment of the imaging system described above, the processor is further configured for configuring the imaging system for the X-ray scatter measurement and the X-ray transmission measurement to occur synchronously.

In an embodiment of the imaging system described above, the processor is further configured for producing a spatially resolved scatter tissue image based on the received X-ray transmission data and the received X-ray scatter data.

In an embodiment of the imaging system described above, the processor is further configured for estimating a spatially resolved X-ray scatter spectral reconstruction based on the received X-ray scatter data, the received X-ray transmission data, and the configuration data.

In an embodiment of the imaging system described above, the X-ray source includes multiple X-ray sources, wherein at least one X-ray source is configurable for the X-ray transmission measurement and at least one X-ray source is configurable for the X-ray scatter measurement.

In an embodiment of the imaging system described above, at least one of the X-ray detecting elements is positioned to detect transmitted X-ray radiation during the X-ray transmission measurement and to detect scattered X-ray radiation during the X-ray scatter measurement.

In an embodiment of the imaging system described above, the processor is further configured for computing an estimate of patient motion based on the received X-ray transmission data, and estimating a spatially resolved X-ray scatter spectral reconstruction based on the received X-ray scatter data and the estimate of patient motion.

In an embodiment of the imaging system described above, the processor is further configured for computing configuration data for the X-ray scatter measurement based on the received X-ray transmission data and an effect of patient motion on the X-ray scatter data, and configuring the imaging system for an X-ray scatter measurement based on the computed configuration data.

In an embodiment of the imaging system described above, the configuration data for the X-ray transmission measurement further comprises more than one orientation of the primary beam about the bore axis, the X-ray transmission measurement comprises measuring the X-rays transmitted directly through the tissue from the primary X-ray beam from more than one orientation about the bore axis, and the processor is further configured for computing an X-ray transmission computed tomography (CT) reconstruction from the received X-ray transmission data from more than one of perspective of the tissue, such that the imaging system operates as an X-ray transmission computed tomography (CT) imaging system.

In an embodiment of the imaging system described above, the X-ray source comprises multiple X-ray sources at different orientations about the bore axis to enable generating a primary X-ray beam from more than one orientation about the bore axis.

In an embodiment of the imaging system described above, the X-ray source is rotatable about the bore axis to enable generating a primary X-ray beam from more than one orientation about the bore axis.

In an embodiment of the imaging system described above, the coded aperture is positioned within the enclosure.

In an embodiment of the imaging system described above, the coded aperture is positioned within the bore.

In an embodiment of the imaging system described above, the coded aperture and the plurality of X-ray detecting elements positioned to detect the scattered X-ray signal are maintained at a location along the bore axis direction in the imaging system such that the primary X-ray beam does not impinge on the coded aperture or the plurality of X-ray detecting elements positioned to detect the scattered X-ray signal during the scatter measurement.

In an embodiment of the imaging system described above, the X-ray source comprises multiple X-ray sources positioned within the imaging system at different locations along the bore axis direction, and at least one X-ray source is configurable for the X-ray transmission measurement and at least one X-ray source is configurable for the X-ray scatter measurement.

In an embodiment of the imaging system described above, the X-ray source comprises multiple X-ray sources positioned within the imaging system at the same location along the bore axis direction, and at least one X-ray source is configurable for the X-ray transmission measurement and at least one X-ray source is configurable for the X-ray scatter measurement.

In an embodiment of the imaging system described above, the X-ray source and a plurality of the X-ray detecting elements are translatable along the bore axis direction and operable while in motion, and the processor is further configured for configuring the X-ray source and plurality of X-ray detecting elements to translate along the bore axis direction during the X-ray transmission measurement.

In an embodiment of the imaging system described above, the coded aperture is moveable to a position between the tissue and the X-ray detector array to modulate the scattered X-ray signal during the X-ray scatter measurement, the coded aperture movement is controllable by the processor, and the configuration data further comprises at least one of timing data, location data, or orientation data for the coded aperture.

In an embodiment of the imaging system described above, at least one of the X-ray source, the collimator, or a plurality of the X-ray detecting elements is moveable and controllable by the processor, and the configuration data further comprises at least one of timing data, location data, or orientation data for the at least one moveable X-ray source, collimator, or plurality of X-ray detecting elements such that the plurality of the X-ray detecting elements is offset at an angle from the primary X-ray beam path for an X-ray scatter measurement to increase the relative solid angular coverage of the X-ray detector array for a range of X-ray scatter angles from a point in the tissue.

In an embodiment of the imaging system described above, the processor is further configured for identifying a region of interest in the patient based on the X-ray radiodensity tissue image, computing configuration data for the X-ray scatter measurement based on the identified region of interest in the patient, and configuring the imaging system for the X-ray scatter measurement based on the computed configuration data.

In an embodiment of the imaging system described above, the processor is further configured for receiving user input selecting the region of interest for the X-ray scatter measurement.

In an embodiment of the imaging system described above, the processor is further configured for transmitting the X-ray radiodensity tissue image to a display, and wherein the user input comprises an indication of a subregion of the X-ray radiodensity tissue image displayed on the display.

In an embodiment of the imaging system described above, the processor is further configured for computing a spatially resolved estimate of a tissue property from the X-ray radiodensity tissue image, and computing a region of interest in the X-ray radiodensity tissue image using the spatially resolved estimate of the tissue property.

In an embodiment of the imaging system described above, the processor is further configured for using a machine learning algorithm to compute the region of interest in the X-ray radiodensity tissue image.

In an embodiment of the imaging system described above, the system further includes a movable beam block configurable to move to a position in the path of the primary X-ray beam between the tissue and the X-ray detector array to block the primary X-ray beam during the X-ray scatter measurement. The beam block is controllable by the processor, and the configuration data further comprises at least one of timing data, location data, or orientation data for the beam block.

In an embodiment of the imaging system described above, the spatially resolved tissue property includes a tissue type that indicates cancerous tissue or benign tissue.

In an embodiment of the imaging system described above, the processor is further configured for reconstructing an estimate of spatially resolved X-ray scatter spectra of the irradiated tissue using the received X-ray scatter data and a forward model of the imaging system, wherein the estimate of spatially resolved X-ray scatter spectra of the irradiated tissue is used to produce the spatially resolved scatter tissue image.

In an embodiment of the imaging system described above, the processor is further configured for selecting an existing forward model of the imaging system using the configuration data or generating a new forward model of the imaging system using the configuration data.

In an embodiment of the imaging system described above, the processor is further configured for computing a spatially resolved estimate of a momentum transfer spectra of the irradiated tissue from the X-ray scatter data, and using the spatially resolved estimate of the momentum transfer spectra of the irradiated tissue to producing a spatially resolved scatter tissue image.

In an embodiment of the imaging system described above, the processor is further configured for using a reference library of existing tissue momentum transfer spectra in combination with the spatially resolved estimate of the momentum transfer spectra of the irradiated tissue to compute the spatially resolved estimate of a tissue property of the irradiated tissue.

In an embodiment of the imaging system described above, the processor is further configured for computing a spatially resolved estimate of a tissue property of the irradiated tissue using a classification algorithm.

In an embodiment of the imaging system described above, the processor is further configured for using a machine learning algorithm for classification in the computation of the spatially resolved estimate of a tissue property.

In an embodiment of the imaging system described above, the processor is further configured for using a rules-based algorithm for classification in the computation of the spatially resolved estimate of a tissue property.

In an embodiment of the imaging system described above, the X-ray source is an X-ray generator, controllable by the processor, and the configuration data further comprises at least one of X-ray source current or X-ray source voltage.

In an embodiment of the imaging system described above, the collimator includes an opening that is configurable in at least one dimension to shape the primary X-ray beam, the at least one dimension of the collimator opening is controllable by the processor, and the configuration data further comprises a dimension of the opening of the collimator.

In an embodiment of the imaging system described above, the system further includes a moveable filter, controllable by the processor, positioned between the X-ray source and the tissue when the X-ray source is irradiating the tissue to modify the energy spectrum and irradiance of the initial X-ray beam. The configuration data further comprises at least one of timing data, location data, or orientation data for the filter.

In an embodiment of the imaging system described above, the tissue to be irradiated is moveable and controllable by the processor, and wherein the configuration data further comprises at least one of timing data, location data, or orientation data for the tissue.

In an embodiment of the imaging system described above, at least one of the coded aperture, the X-ray source, the collimator, or a plurality of the X-ray detecting elements is moveable and controllable by the processor, wherein the configuration data further comprises at least one of timing data, location data, or orientation data for the at least one moveable X-ray source, collimator, coded aperture, or plurality of X-ray detecting elements.

In an embodiment of the imaging system described above, at least one of the X-ray source or a plurality of the X-ray detecting elements is moveable and controllable by the processor, wherein the configuration data further comprises at least one of timing data, location data, or orientation data for the at least one moveable X-ray source or plurality of X-ray detecting elements, and wherein the at least one moveable X-ray source or plurality of X-ray detecting elements is operable while in motion.

In an embodiment of the imaging system described above, the configuration data for the X-ray scatter measurement further comprises more than one orientation of the primary beam about the bore axis, the X-ray scatter measurement comprises measuring scattered X-ray radiation from the primary X-ray beam passing through the tissue from more than one orientation about the bore axis, and the processor is further configured for estimating the spatially resolved X-ray scatter spectral reconstruction of the tissue based on the received X-ray scatter data from more than one perspective of the tissue.

In an embodiment of the imaging system described above, the processor is further configured for computing an estimate of a data quality metric for the resulting X-ray scatter data from the configuration data; and computing optimized configuration data using the estimated X-ray scatter data quality metric.

In an embodiment of the imaging system described above, the processor is further configured for computing an estimate of radiation dose to the patient for an X-ray scatter measurement from the configuration data; computing an estimate of a data quality metric for the resulting X-ray scatter data from the configuration data; and computing optimized configuration data using the estimated radiation dose and the estimated X-ray scatter data quality metric.

In an embodiment of the imaging system described above, the system further includes a camera for recording the patient. The the processor is further configured for receiving recording data from the camera, computing an estimate of patient motion based on the received recording data, and estimating a spatially resolved X-ray scatter spectral reconstruction based on the received X-ray scatter data and the estimate of patient motion.

In an embodiment of the imaging system described above, the processor is further configured for computing configuration data for the X-ray scatter measurement based on the received recording data. The configuration data is computed based on an effect of patient motion on the X-ray scatter data.

In an embodiment of the imaging system described above, the coded aperture includes a periodic pattern in at least one dimension having a single attenuating component with openings focused at a millimeter-scale focal point between the coded aperture and the X-ray source, the coded aperture is moveable and controllable by the processor, the tissue to be irradiated is moveable and controllable by the processor, and the configuration data further comprises at least one of timing data for the tissue, location data for the tissue, orientation data for the tissue, timing data for the coded aperture, location data for the coded aperture, or orientation data for the coded aperture to direct the focal point of the coded aperture at a point in the tissue.

In an embodiment of the imaging system described above, the coded aperture comprises a set of moveable and controllable attenuating components arranged in a periodic pattern in at least one dimension, the tissue to be irradiated is moveable and controllable by the processor, and the configuration data further comprises at least one of timing data for the tissue, location data for the tissue, orientation data for the tissue, timing data for the attenuating components, location data for the attenuating components, or orientation data for the attenuating components to focus the coded aperture at a millimeter-scale focal point between the coded aperture and the X-ray source and direct the focal point at a point in the tissue.

In an embodiment of the imaging system described above, the system further includes a secondary collimator positioned between the tissue and the X-ray detector array to collimate the scattered X-ray radiation or a filter positioned between the tissue and the X-ray detector array to modify the energy spectrum and irradiance of the scattered X-ray radiation.

In an embodiment of the imaging system described above, the system further includes a secondary coded aperture positioned between the X-ray source and the tissue configured for modulating the initial X-ray beam.

In another aspect, an imaging system for performing in vivo imaging of a human body is disclosed. The imaging system includes an X-ray source mounted to a configurable arm for irradiating in vivo tissue of at least a portion of the body with a primary X-ray beam. The position and orientation of the X-ray source is adjustable by the user. The imaging system further includes a collimator positioned between the X-ray source and the tissue to shape the primary X-ray beam. The imaging system further includes an X-ray detector array comprising a plurality of X-ray detecting elements arranged in at least two dimensions. The at least one of the X-ray detecting elements is positioned distally from the X-ray source outside the path of the primary X-ray beam past the irradiated portion of the body to measure scattered X-ray radiation from the primary X-ray beam passing through the tissue. The imaging system further includes a coded aperture positioned between the tissue and the X-ray detector array. The coded aperture is configured to modulate the scattered X-ray radiation from the tissue detected by the X-ray detector array. The imaging system further includes a control system comprising memory and a processor. The processor is configured for determining configuration data for the imaging system. The configuration data comprises the location and orientation of the X-ray source. The processor is further configured for performing the X-ray scatter measurement with the configured imaging system. The processor is further configured for receiving data representing scattered X-ray radiation detected by the X-ray detector array. The processor is further configured for estimating a spatially resolved X-ray scatter spectral reconstruction of the tissue based on the received X-ray scatter data and the configuration data. The processor is further configured for determining a spatially resolved tissue property based on the received X-ray scatter data. The processor is further configured for producing a spatially resolved scatter tissue image based on the received X-ray scatter data.

In an embodiment of the imaging system described above, the configurable arm is a C-arm with the X-ray source mounted proximate to a first end of the C-arm and at least one of the X-ray detecting elements mounted proximate to a second end of the C-arm. The C-arm is adjustable by the user such that a least some portion of the body is positioned between the X-ray source and the at least one detecting element for the X-ray scatter measurement.

In an embodiment of the imaging system described above, the configurable arm is mounted to a ceiling, floor, wall, or other fixed surface, or is mounted to a mobile carriage, wherein the carriage can be positioned by the user.

In an embodiment of the imaging system described above, at least one X-ray detecting element is positioned to detect X-rays transmitted directly through the tissue from the initial primary X-ray beam such that the imaging system operates as an X-ray transmission imaging system. The processor is further configured for performing the X-ray transmission measurement with the configured imaging system, receiving data representing transmitted X-ray radiation detected by the X-ray detector array, and computing an X-ray radiodensity tissue image from the received X-ray transmission data.

In an embodiment of the imaging system described above, the processor is further configured for determining the location and orientation of the X-ray source based on the received X-ray transmission data.

According to another aspect, a method for performing in vivo tissue imaging of a human body is disclosed. According to one embodiment, the method comprises positioning at least a portion of the body in an imaging system. The method further comprises configuring the imaging system for an X-ray scatter measurement based on configuration data that comprises at least one of timing data, location data, and orientation data for an X-ray source and a plurality of X-ray detecting elements configured to rotate about a bore axis of the body. The method further comprises performing the X-ray scatter measurement with the configured imaging system. The X-ray scatter measurement comprises irradiating in vivo tissue with an initial X-ray beam from the X-ray source through a collimator configured to rotate about the bore axis between the X-ray source and the tissue to direct the initial X-ray beam. The X-ray scatter measurement further comprises modulating scattered X-ray radiation from the tissue using a coded aperture configured to rotate about the bore axis between the tissue and an X-ray detector array comprising an arrangement of X-ray detecting elements in at least two dimensions. The X-ray scatter measurement further comprises detecting the modulated scattered X-ray radiation signal from the tissue with a plurality of the X-ray detecting elements configured to rotate about the bore axis and positioned to detect scattered X-ray radiation. The X-ray scatter measurement further comprises receiving data representing the detected scattered X-ray radiation from the X-ray detector array. The method further comprises analyzing the received data to generate a representation of the irradiated tissue from the received data based on the configuration data. The representation comprises a spatially resolved property of the irradiated tissue.

According to another aspect, a method for in vivo tissue imaging is disclosed. According to one embodiment, the method comprises configuring an imaging system for an X-ray scatter measurement based on configuration data that comprises at least one of timing data, location data, and orientation data for an X-ray source and a plurality of X-ray detecting elements. The method further comprises enacting the X-ray scatter measurement with the configured imaging system. The X-ray scatter measurement comprises irradiating in vivo tissue with an initial X-ray beam from the X-ray source through a collimator disposed between the X-ray source and the tissue to direct the initial X-ray beam. The X-ray scatter measurement further comprises modulating scattered X-ray radiation from the tissue using a coded aperture disposed between the tissue and an X-ray detector array comprising an arrangement of X-ray detecting elements in at least two dimensions. The X-ray scatter measurement further comprises detecting the modulated scattered X-ray radiation signal from the tissue with a plurality of the X-ray detecting elements disposed to detect scattered X-ray radiation. The X-ray scatter measurement further comprises receiving data representing the detected scattered X-ray radiation from the X-ray detector array. The method further comprises analyzing the received data to generate a representation of the irradiated tissue. The representation is generated from the received data. The representation is based on the configuration data. The representation comprises a spatially resolved property of the irradiated tissue.

In an embodiment of the method described above, the representation of the irradiated tissue comprises indications of potentially cancerous regions within the irradiated tissue.

In an embodiment of the method described above, an estimate of spatially resolved X-ray scatter spectra of the irradiated tissue is reconstructed using the received X-ray scatter data and a forward model of the in vivo tissue imaging system. In the embodiment, of the method the estimate of spatially resolved X-ray scatter spectra of the irradiated tissue is used to generate the representation of the irradiated tissue.

An embodiment of the method described above further comprises selecting an existing forward model of the in vivo tissue imaging system using the configuration data or generating a new forward model of the in vivo tissue imaging system using the configuration data.

An embodiment of the method described above further comprises computing a spatially resolved estimate of the momentum transfer spectra of the irradiated tissue from the X-ray scatter data. The embodiment further comprises computing a spatially resolved estimate of a tissue property of the irradiated tissue using the spatially resolved estimate of the momentum transfer spectra of the irradiated tissue.

In an embodiment of the method described above, a reference library of existing tissue momentum transfer spectra is used in combination with the spatially resolved estimate of the momentum transfer spectra of the irradiated tissue to compute the spatially resolved estimation of a tissue property of the irradiated tissue.

In an embodiment of the method described above, classification algorithms are used to compute the spatially resolved estimation of a tissue property of the irradiated tissue.

In an embodiment of the method described above, machine learning algorithms are used for classification in the computation of the spatially resolved estimation of a tissue property.

In an embodiment of the method described above, rules-based algorithms are used for classification in the computation of the spatially resolved estimation of a tissue property.

In an embodiment of the method described above, at least one of the coded aperture, the X-ray source, the collimator, or a plurality of the X-ray detecting elements is moveable. The embodiment further comprises moving the at least one moveable coded aperture, X-ray source, collimator, or plurality of X-ray detecting elements prior to an X-ray scatter measurement. The embodiment further comprises receiving configuration data that comprises at least one of location data or orientation data for the at least one moveable coded aperture, X-ray source, collimator, or plurality of X-ray detecting elements.

In an embodiment of the method described above, the X-ray source is an X-ray generator, controllable by the processor. In the embodiment, the configuration data further comprises at least one of X-ray source current or X-ray source voltage. The embodiment further comprises configuring the X-ray source current or X-ray source voltage for the X-ray scatter measurement.

In an embodiment of the method described above, the collimator includes a controllable opening. In the embodiment, the configuration data further comprises at least one of timing data for the controllable opening, size of the collimator opening, or shape of the collimator opening. The embodiment further comprises at least one of controlling the spatial extent of the initial X-ray beam by configuring the size of the collimator opening for the X-ray scatter measurement or controlling the cross-sectional shape of the initial X-ray beam by configuring the shape of the collimator opening for the X-ray scatter measurement.

In an embodiment of the method described above, the configuration data further comprises at least one of timing data, location data, or orientation data for a moveable and controllable filter. The embodiment further comprises modifying the energy spectrum and irradiance of the initial X-ray beam by configuring the location or the orientation of the moveable filter to dispose the filter between the X-ray source and the tissue for the X-ray scatter measurement.

In an embodiment of the method described above, the tissue to be irradiated is moveable and controllable. In the embodiment, the configuration data further comprises at least one of timing data, location data, or orientation data for the tissue. The embodiment further comprises configuring at least one of the location or the orientation of the tissue for the X-ray scatter measurement.

In an embodiment of the method described above, at least one of the coded aperture, the X-ray source, the collimator, or a plurality of the X-ray detecting elements is moveable and controllable. In the embodiment, the configuration data further comprises at least one of timing data, location data, or orientation data for the at least one moveable coded aperture, X-ray source, collimator, or plurality of X-ray detecting elements. The embodiment further comprises configuring at least one of the location or the orientation of the at least one moveable coded aperture, X-ray source, collimator, or plurality of X-ray detecting elements for the X-ray scatter measurement.

In an embodiment of the method described above, at least one of the X-ray source or a plurality of the X-ray detecting elements is moveable and controllable. In the embodiment, the configuration data further comprises at least one of timing data, location data, or orientation data for the at least one moveable X-ray source or plurality of X-ray detecting elements. In the embodiment, the at least one moveable X-ray source or plurality of X-ray detecting elements is operable while in motion. The embodiment further comprises configuring the at least one moveable X-ray source or plurality of X-ray detecting elements to be in motion during the X-ray scatter measurement.

An embodiment of the method described above further comprises controlling the perspective of the tissue for the X-ray scatter measurement by configuring at least one of the location or the orientation of the at least one moveable X-ray source, collimator, coded aperture, or plurality of X-ray detecting elements. The embodiment further comprises performing a plurality of X-ray scatter measurements from different perspectives of the tissue. The embodiment further comprises analyzing the received data from the plurality of different perspectives to generate the representation of the irradiated tissue.

In an embodiment of the method described above, the at least one moveable X-ray source, collimator, coded aperture, or plurality of X-ray detecting elements is rotatable about the tissue. In the embodiment, configuring at least one of the location or the orientation of the at least one rotatable X-ray source, collimator, coded aperture, or plurality of X-ray detecting elements further comprises rotating the at least one moveable X-ray source, collimator, coded aperture, or plurality of X-ray detecting elements about the tissue.

An embodiment of the method described above further comprises computing an estimate of a data quality metric for the resulting X-ray scatter data from the configuration data computing optimized configuration data using the estimated X-ray scatter data quality metric. In the embodiment, the optimized configuration data is computed to define a perspective for an X-ray scatter measurement of a point in the tissue to satisfy at least one of minimizing the attenuation of the initial X-ray beam along the initial X-ray beam path to the point in the tissue or minimizing the attenuation of the scattered X-ray signal from the point in the tissue to an X-ray detecting element.

An embodiment of the method described above further comprises computing an estimate of radiation dose to the patient for an X-ray scatter measurement from the configuration data. The embodiment further comprises computing an estimate of a data quality metric for the resulting X-ray scatter data from the configuration data. The embodiment further comprises computing optimized configuration data using the estimated radiation dose and the estimated X-ray scatter data quality metric. The embodiment further comprises configuring the imaging system for an X-ray scatter measurement based on the computed configuration data.

An embodiment of the method described above further comprises recording the patient with a camera. The embodiment further comprises receiving camera recording data. The embodiment further comprises analyzing the recording data to compute an estimate of patient motion. The embodiment further comprises using the computed estimate of patient motion in combination with received X-ray scatter data to generate a representation of the irradiated tissue that accounts for an effect of patient motion.

In an embodiment of the method described above, at least one of the X-ray source, the collimator, the coded aperture, or a plurality of the X-ray detecting elements is further configurable. The embodiment further comprises analyzing the estimate of patient motion to compute configuration data for the at least one further configurable X-ray source, collimator, coded aperture, or plurality of X-ray detecting elements. In the embodiment, the configuration data is computed to minimize an effect of patient motion on the X-ray scatter data. The embodiment further comprises configuring the imaging system for an X-ray scatter measurement based on the computed configuration data.

An embodiment of the method described above further comprises blocking the initial X-ray beam with a beam block disposed in the path of the initial X-ray beam between the tissue and the X-ray detector array.

In an embodiment of the method described above, the coded aperture includes a periodic pattern in at least one dimension having a single attenuating component with openings focused at a millimeter-scale focal point 100-2000 millimeters from the coded aperture. In the embodiment, the coded aperture is moveable and controllable. In the embodiment, the tissue to be irradiated is moveable and controllable. In the embodiment, the configuration data further comprises at least one of timing data for the tissue, location data for the tissue, orientation data for the tissue, timing data for the coded aperture, location data for the coded aperture, or orientation data for the coded aperture. The embodiment further comprises directing the focal point of the coded aperture at a point in the tissue for the X-ray scatter measurement by configuring at least one of the location of the coded aperture, the orientation of the coded aperture, the location of the tissue, or the orientation of the tissue.

In an embodiment of the method described above, the coded aperture comprises a set of moveable and controllable attenuating components arranged in a periodic pattern in at least one dimension. In the embodiment, the tissue to be irradiated is moveable and controllable by the processor. In the embodiment, the configuration data further comprises at least one of timing data for the tissue, location data for the tissue, orientation data for the tissue, timing data for the attenuating components, location data for the attenuating components, or orientation data for the attenuating components. The embodiment further comprises focusing the coded aperture at a millimeter-scale focal point 100-2000 millimeters from the coded aperture and directing the focal point at a point in the tissue for the X-ray scatter measurement by configuring at least one of the location of the attenuating components, the orientation of the attenuating components, the location of the tissue, or the orientation of the tissue.

An embodiment of the method described above further comprises transmitting the representation of the irradiated tissue to a display.

An embodiment of the method described above further comprises at least one of collimating the scattered X-ray radiation with a secondary collimator disposed between the tissue and the X-ray detector array or modifying the energy spectrum and irradiance of the scattered X-ray radiation with a filter disposed between the tissue and the X-ray detector array.

An embodiment of the method described above further comprises modulating the initial X-ray beam with a secondary coded aperture disposed between the X-ray source and the tissue.

An embodiment of the method described above further comprises configuring the imaging system for an X-ray transmission measurement based on the configuration data. In the embodiment, the configuration data further comprises at least one of timing data, location data, or orientation data for the X-ray source and a plurality of the X-ray detecting elements. The embodiment further comprises performing the X-ray transmission measurement with the configured imaging system. The X-ray transmission measurement comprises irradiating in vivo tissue with an initial X-ray beam from the X-ray source through the collimator. The X-ray transmission measurement further comprises detecting X-ray radiation transmitted directly through the tissue with a plurality of the X-ray detecting elements positioned to detect X-rays transmitted directly through the tissue from the initial X-ray beam such that the imaging system operates as an X-ray transmission imaging system. The X-ray transmission measurement further comprises receiving data representing the detected transmitted X-ray radiation from the X-ray detector array. The embodiment further comprises computing X-ray radiodensity tissue images from the X-ray transmission data.

In an embodiment of the method described above, the configuration data for an X-ray transmission measurement is different from the configuration data for an X-ray scatter measurement.

In an embodiment of the method described above, the coded aperture is moveable and controllable by the processor. In the embodiment, the coded aperture is moveable to a position between the tissue and the X-ray detector array to modulate the scattered X-ray signal during an X-ray scatter measurement. In the embodiment, the configuration data further comprises at least one of timing data, location data, or orientation data for the coded aperture. The embodiment further comprises modulating the scattered X-ray signal by configuring at least one of the location or orientation of the coded aperture such that the coded aperture is positioned between the tissue and the X-ray detector array.

In an embodiment of the method described above, at least one of the X-ray source, the collimator, or a plurality of the X-ray detecting elements is moveable and controllable. In the embodiment, the configuration data further comprises timing data, location data, or orientation data for the at least one moveable X-ray source, collimator, or plurality of X-ray detecting elements. The embodiment further comprises offsetting a plurality of the X-ray detecting elements at an angle from the initial X-ray beam path during the X-ray scatter measurement by configuring at least one of the orientation or location of the at least one moveable X-ray source, collimator, or plurality of X-ray detecting elements to increase the relative solid angular coverage of the X-ray detector array for a range of X-ray scatter angles from a point in the tissue.

In an embodiment of the method described above, at least one of the X-ray source, the collimator, the coded aperture, or a plurality of the X-ray detecting elements is further configurable. In the embodiment, the configuration data specifies a configuration for an X-ray scatter measurement subsequent to an X-ray transmission measurement such that the tissue volume imaged during the subsequent X-ray scatter measurement is a subregion of the tissue volume imaged during the X-ray transmission measurement.

An embodiment of the method described above further comprises receiving user input for selecting a subregion of tissue for the X-ray scatter measurement. The embodiment further comprises computing configuration data for the at least one further configurable X-ray source, collimator, coded aperture, or plurality of X-ray detecting elements using the user input. The embodiment further comprises configuring the imaging system for an X-ray scatter measurement on the selected subregion of tissue based on the computed configuration data.

An embodiment of the method described above further comprises transmitting the X-ray radiodensity tissue image to a display. In the embodiment, the user input comprises an indication of a subregion of the displayed tissue image.

An embodiment of the method described above further comprises computing a spatially resolved estimate of the likelihood of cancer from the X-ray radiodensity tissue image. The embodiment further comprises computing regions of interest in the displayed X-ray radiodensity tissue image using the spatially resolved estimate of the likelihood of cancer. The embodiment further comprises transmitting the region of interest data to the display.

An embodiment of the method described above further comprises using machine learning algorithms to compute regions of interest in the displayed X-ray radiodensity tissue image.

In an embodiment of the method described above, the configuration data further comprises at least one of timing data, location data, or orientation data for a moveable and controllable beam block. In the embodiment, the beam block is moveable to a position in which the beam block is disposed in the path of the initial X-ray beam between the tissue and the X-ray detector array to block the initial X-ray beam during an X-ray scatter measurement. The embodiment further comprises blocking the initial X-ray beam by configuring at least one of the location or orientation of the beam block to dispose the beam block in the initial X-ray beam path between the tissue and the X-ray detector array for the X-ray scatter measurement.

An embodiment of the method described above further comprises configuring the imaging system for an X-ray scatter measurement and an X-ray transmission measurement that occur synchronously.

An embodiment of the method described above further comprises overlaying the representation of the tissue generated from the X-ray scatter data with the X-ray radiodensity tissue image computed from X-ray transmission data. The embodiment further comprises transmitting the overlaid representation and image to a display.

An embodiment of the method described above further comprises analyzing the received X-ray transmission data in combination with the received X-ray scatter data to generate the representation of the irradiated tissue.

In an embodiment of the method described above the X-ray source includes multiple X-ray sources. In the embodiment, at least one X-ray source is configured for X-ray transmission measurements and at least one X-ray source is configured for X-ray scatter measurements. The embodiment further comprises performing an X-ray transmission measurement using the X-ray source configured for X-ray transmission measurements and performing an X-ray scatter measurement using the X-ray source configured for X-ray transmission measurements.

An embodiment of the method described above further comprises configuring at least one of the X-ray detecting elements for detecting transmitted X-ray radiation during the X-ray transmission measurement and for detecting scattered X-ray radiation during the X-ray scatter measurement.

An embodiment of the method described above further comprises analyzing the received X-ray transmission data to compute an estimate of patient motion. The embodiment further comprises using the estimate of patient motion in combination with the received X-ray scatter data to generate a representation of the irradiated tissue that accounts for an effect of patient motion.

In an embodiment of the method described above, at least one of the X-ray source, the collimator, the coded aperture, or a plurality of the X-ray detecting elements is further configurable. The embodiment further comprises analyzing X-ray transmission data to compute configuration data for the at least one further configurable X-ray source, collimator, coded aperture, or plurality of X-ray detecting elements. In the embodiment, the configuration data is computed to minimize an effect of patient motion on the X-ray scatter data. The embodiment further comprises configuring the imaging system for an X-ray scatter measurement based on the computed configuration data.

In an embodiment of the method described above, at least one of the X-ray source, the collimator, or a plurality of the X-ray detecting elements is moveable and controllable. In the embodiment, the configuration data further comprises at least one of timing data, location data, or orientation data for the at least one moveable X-ray source, collimator, or plurality of the X-ray detecting elements. The embodiment further comprises controlling the perspective of the tissue for the X-ray transmission measurement by configuring at least one of the location or the orientation of the at least one moveable X-ray source, collimator, or plurality of X-ray detecting elements. The embodiment further comprises performing a plurality of X-ray transmission measurements from different perspectives of the tissue. The embodiment further comprises computing X-ray transmission computed tomography (CT) reconstructions from the received X-ray transmission data from a plurality of perspectives of the tissue, such that the imaging system operates as an X-ray transmission computed tomography (CT) imaging system.

In an embodiment of the method described above, at least one of the X-ray source or the X-ray detector is rotatable about the tissue and controllable. In the embodiment, the configuration data further comprises at least one of timing data, location data, or orientation data for the at least one rotatable X-ray source or plurality of X-ray detecting elements. The embodiment further comprises controlling the perspective of the tissue for the plurality of X-ray transmission measurements from different perspectives by configuring the orientation of the at least one rotatable X-ray source or plurality of X-ray detecting elements about the tissue.

In an embodiment of the method described above, the X-ray source and a plurality of the X-ray detecting elements arc rotatable about the tissue and controllable. In the embodiment, the configuration data further comprises at least one of timing data, location data, or orientation data for the rotatable X-ray source and plurality of X-ray detecting elements. In the embodiment, the rotatable X-ray source and plurality of X-ray detecting elements are attached to and rotate with a rotatable gantry in a gantry enclosure around a bore in which the tissue is located. The embodiment further comprises controlling the perspective of the tissue for the plurality of X-ray transmission measurements from different perspectives by configuring the orientation of the rotatable X-ray source and plurality of X-ray detecting elements on the gantry about the tissue.

In an embodiment of the method described above, the coded aperture is disposed within the gantry enclosure of the X-ray transmission computed tomography (CT) imaging system, or is disposed within the bore of the X-ray transmission computed tomography (CT) imaging system.

In an embodiment of the method described above, the coded aperture and the plurality of X-ray detecting elements disposed to detect the scattered X-ray signal are maintained at a location along the bore axis direction in the X-ray transmission computed tomography (CT) imaging system such that the initial X-ray beam does not impinge on the coded aperture or the plurality of X-ray detecting elements disposed to detect the scattered X-ray signal.

In an embodiment of the method described above, the X-ray source includes multiple X-ray sources, with the X-ray sources disposed within the X-ray transmission computed tomography (CT) imaging system at different locations along the bore axis. In the embodiment, at least one X-ray source is configured for X-ray transmission measurements and at least one X-ray source is configured for X-ray scatter measurements. The embodiment further comprises performing an X-ray transmission measurement using the X-ray source configured for X-ray transmission measurements and performing an X-ray scatter measurement using the X-ray source configured for X-ray transmission measurements.

In an embodiment of the method described above, the X-ray source includes multiple X-ray sources, with the X-ray sources disposed within the X-ray transmission computed tomography (CT) imaging system at the same location along the bore axis. In the embodiment, at least one X-ray source is configured for X-ray transmission measurements and at least one X-ray source is configured for X-ray scatter measurements. The embodiment further comprises performing an X-ray transmission measurement using the X-ray source configured for X-ray transmission measurements and performing an X-ray scatter measurement using the X-ray source configured for X-ray transmission measurements.

In an embodiment of the method described above, the gantry is translatable along the bore axis direction with the attached X-ray source and plurality of X-ray detecting elements. In the embodiment, the rotatable X-ray source and plurality of X-ray detecting elements are translatable along the bore axis direction and controllable. In the embodiment, the configuration data further comprises at least one of location data for the rotatable and translatable X-ray source and plurality of X-ray detecting elements. In the embodiment, the X-ray source and plurality of X-ray detecting elements are operable while in motion. The embodiment further comprises configuring the rotatable and translatable X-ray source and plurality of X-ray detecting elements to be in motion while the gantry is simultaneously rotating about the bore axis direction and translating along the bore axis direction during the X-ray transmission measurement.

An embodiment of the method described above is performed in a C-arm X-ray imaging system, a ceiling mounted X-ray imaging system, or a portable/mobile X-ray imaging system.

In one embodiment, a control system for an in vivo tissue imaging system for performing in vivo imaging of a human body is disclosed. The control system includes a memory and a processor. The processor is configured for configuring the imaging system for an X-ray scatter measurement based on configuration data comprising at least one of timing data, location data, and orientation data for an X-ray source and a plurality of X-ray detecting elements for the X-ray scatter measurement. The processor is further configured for performing the X-ray scatter measurement with the configured imaging system. The X-ray scatter measurement comprises irradiating in vivo tissue with an initial X-ray beam from the X-ray source through a collimator positioned between the X-ray source and the tissue to direct the initial X-ray beam. The X-ray scatter measurement further comprises modulating scattered X-ray radiation from the tissue using a coded aperture positioned between the tissue and an X-ray detector array comprising an arrangement of X-ray detecting elements in at least two dimensions. The X-ray scatter measurement further comprises detecting the modulated scattered X-ray radiation signal from the tissue with a plurality of the X-ray detecting elements positioned to detect scattered X-ray radiation. The X-ray scatter measurement further comprises receiving data representing the scattered X-ray radiation detected by the X-ray detector array with the processor. The processor is further configured for analyzing the received data to generate a representation of the irradiated tissue from the received data based on the configuration data, wherein the representation comprises a spatially resolved property of the irradiated tissue.

In an embodiment of the control system for an in vivo tissue imaging system disclosed above, a plurality of the X-ray detecting elements are positioned to detect X-rays transmitted directly through the tissue from the initial X-ray beam such that the imaging system operates as an X-ray transmission imaging system. The processor is further configured for configuring the imaging system for an X-ray transmission measurement based on the configuration data. The configuration data further comprises at least one of timing data, location data, and orientation data for the X-ray source and a plurality of the X-ray detecting elements for the X-ray transmission measurement. The processor is further configured for performing the X-ray transmission measurement with the configured imaging system. The processor is further configured for: receiving data representing transmitted X-ray radiation detected by the X-ray detector array. The processor is further configured for computing X-ray radiodensity tissue images from the received X-ray transmission data.

In an embodiment of the control system for an in vivo tissue imaging system disclosed above, the configuration data for the X-ray transmission measurement is different from the configuration data for the X-ray scatter measurement.

In an embodiment of the control system for an in vivo tissue imaging system disclosed above, the coded aperture is moveable and controllable by the processor. The coded aperture is moveable to a position in which the coded aperture is between the tissue and the X-ray detector array to modulate the scattered X-ray signal during the X-ray scatter measurement. The configuration data further comprises at least one of timing data, location data, or orientation data for the coded aperture.

In an embodiment of the control system for an in vivo tissue imaging system disclosed above, at least one of the X-ray source, the collimator, or a plurality of the X-ray detecting elements is moveable and controllable by the processor. The configuration data further comprises at least one of timing data, location data, or orientation data for the at least one moveable X-ray source, collimator, or plurality of X-ray detecting elements such that a plurality of the X-ray detecting elements is offset at an angle from the initial X-ray beam path for the X-ray scatter measurement to increase the relative solid angular coverage of the X-ray detector array for a range of X-ray scatter angles from a point in the tissue.

In an embodiment of the control system for an in vivo tissue imaging system disclosed above, the configuration data specifies a configuration for the X-ray scatter measurement subsequent to the X-ray transmission measurement such that the tissue volume imaged during the subsequent X-ray scatter measurement is a subregion of the tissue volume imaged during the X-ray transmission measurement.

In an embodiment of the control system for an in vivo tissue imaging system disclosed above, the processor is further configured for receiving user input selecting a subregion of tissue for the X-ray scatter measurement. The processor is further configured for computing the configuration data for the X-ray source, collimator, coded aperture, or plurality of X-ray detecting elements using the user input. The processor is further configured for configuring the imaging system for the X-ray scatter measurement based on the computed configuration data.

In an embodiment of the control system for an in vivo tissue imaging system disclosed above, the processor is further configured for transmitting the X-ray radiodensity tissue image to a display, and wherein the user input comprises an indication of a subregion of the displayed X-ray radiodensity tissue image.

In an embodiment of the control system for an in vivo tissue imaging system disclosed above, the processor is further configured for computing a spatially resolved estimate of the likelihood of cancer from the X-ray radiodensity tissue image. The processor is further configured for computing regions of interest in the X-ray radiodensity tissue image using the spatially resolved estimate of the likelihood of cancer. The processor is further configured for transmitting the region of interest data to the display.

In an embodiment of the control system for an in vivo tissue imaging system disclosed above, the processor is further configured for using a machine learning algorithm to compute the regions of interest in the X-ray radiodensity tissue image.

In an embodiment of the control system for an in vivo tissue imaging system disclosed above, the configuration data further comprises at least one of timing data, location data, or orientation data for a beam block configured to rotate about the bore axis to a position in the path of the initial X-ray beam between the tissue and the X-ray detector array to block the initial X-ray beam during the X-ray scatter measurement.

In an embodiment of the control system for an in vivo tissue imaging system disclosed above, the processor is further configured for configuring the imaging system for the X-ray scatter measurement and the X-ray transmission measurement to occur synchronously.

In an embodiment of the control system for an in vivo tissue imaging system disclosed above, the processor is further configured for overlaying the representation of the tissue generated from the X-ray scatter data with the X-ray radiodensity tissue image computed from the X-ray transmission data. The processor is further configured for transmitting the overlaid representation and image to a display.

In an embodiment of the control system for an in vivo tissue imaging system disclosed above, the processor is further configured for analyzing the received X-ray transmission data in combination with the received X-ray scatter data to generate the representation of the irradiated tissue.

In an embodiment of the control system for an in vivo tissue imaging system disclosed above, the processor is further configured for analyzing the received X-ray transmission data to compute an estimate of patient motion. The processor is further configured for using the estimate of patient motion in combination with the received X-ray scatter data to generate a representation of the irradiated tissue that accounts for an effect of patient motion.

In an embodiment of the control system for an in vivo tissue imaging system disclosed above, the processor is further configured for analyzing the received X-ray transmission data to compute configuration data for the X-ray source, collimator, coded aperture, or plurality of X-ray detecting elements, wherein the configuration data is computed to minimize an effect of patient motion on the X-ray scatter data. The processor is further configured for configuring the imaging system for the X-ray scatter measurement based on the computed configuration data.

In an embodiment of the control system for an in vivo tissue imaging system disclosed above, the configuration data further comprises at least one of timing data, location data, or orientation data for at least one of the X-ray source, collimator, or plurality of X-ray detecting elements to enable a plurality of different perspectives of the tissue for the X-ray transmission measurement. The processor is further configured for computing X-ray transmission computed tomography (CT) reconstructions from the received X-ray transmission data from a plurality of perspectives of the tissue, such that the imaging system operates as an X-ray transmission computed tomography (CT) imaging system.

In an embodiment of the control system for an in vivo tissue imaging system disclosed above, the configuration data further comprises at least one of timing data, location data, or orientation data for the X-ray source or plurality of X-ray detecting elements to enable a plurality of different perspectives of the tissue for the X-ray transmission measurement.

In an embodiment of the control system for an in vivo tissue imaging system disclosed above, the X-ray source and a plurality of the X-ray detecting elements are rotatable about the tissue and controllable by the processor. The configuration data further comprises at least one of timing data, location data, or orientation data for the X-ray source and plurality of X-ray detecting elements to enable a plurality of different perspectives of the tissue for the X-ray transmission measurement. The X-ray source and plurality of X-ray detecting elements are attached to and rotate with a rotatable gantry in a gantry enclosure around a bore in which the tissue is positioned.

In an embodiment of the control system for an in vivo tissue imaging system disclosed above, the gantry is translatable along the bore axis direction, the X-ray source and plurality of X-ray detecting elements attached to the gantry are translatable along the bore axis direction, the X-ray source and plurality of X-ray detecting elements attached to the gantry are operable while in motion, and the processor is further configured for configuring X-ray source and plurality of X-ray detecting elements to be in motion during the X-ray transmission measurement while the gantry is simultaneously rotating about the bore axis and translating along the bore axis direction.

In an embodiment of the control system for an in vivo tissue imaging system disclosed above, the representation of the irradiated tissue includes indications of potentially cancerous regions within the irradiated tissue.

In an embodiment of the control system for an in vivo tissue imaging system disclosed above, the processor is further configured for reconstructing an estimate of spatially resolved X-ray scatter spectra of the irradiated tissue using the received X-ray scatter data and a forward model of the imaging system, wherein the estimate of spatially resolved X-ray scatter spectra of the irradiated tissue is used to generate the representation of the irradiated tissue.

In an embodiment of the control system for an in vivo tissue imaging system disclosed above, the processor is further configured for selecting an existing forward model of the imaging system using the configuration data or generating a new forward model of the imaging system using the configuration data.

In an embodiment of the control system for an in vivo tissue imaging system disclosed above, the processor is further configured for computing a spatially resolved estimate of the momentum transfer spectra of the irradiated tissue from the X-ray scatter data. The processor is further configured for computing a spatially resolved estimate of a tissue property of the irradiated tissue using the spatially resolved estimate of the momentum transfer spectra of the irradiated tissue.

In an embodiment of the control system for an in vivo tissue imaging system disclosed above, the processor is further configured for using a reference library of existing tissue momentum transfer spectra in combination with the spatially resolved estimate of the momentum transfer spectra of the irradiated tissue to compute the spatially resolved estimate of a tissue property of the irradiated tissue.

In an embodiment of the control system for an in vivo tissue imaging system disclosed above, the processor is further configured for using a classification algorithm to compute the spatially resolved estimate of a tissue property of the irradiated tissue.

In an embodiment of the control system for an in vivo tissue imaging system disclosed above, the processor is further configured for using a machine learning algorithm for classification in the computation of the spatially resolved estimate of a tissue property.

In an embodiment of the control system for an in vivo tissue imaging system disclosed above, the processor is further configured for using a rules-based algorithm for classification in the computation of the spatially resolved estimate of a tissue property.

In an embodiment of the control system for an in vivo tissue imaging system disclosed above, the processor is further configured for receiving configuration data comprising at least one of location data or orientation data for at least one of the coded aperture, X-ray source, collimator, or plurality of X-ray detecting elements.

In an embodiment of the control system for an in vivo tissue imaging system disclosed above, the X-ray source is an X-ray generator, controllable by the processor, and the configuration data further comprises at least one of X-ray source current or X-ray source voltage.

In an embodiment of the control system for an in vivo tissue imaging system disclosed above, the collimator includes a controllable opening, controllable by the processor, and the configuration data further comprises at least one of timing data for the opening, size of the opening to configure spatial extent of the initial X-ray beam, or shape of the opening to configure cross-sectional shape of the initial X-ray beam.

In an embodiment of the control system for an in vivo tissue imaging system disclosed above, the configuration data further comprises at least one of timing data, location data, or orientation data for a, wherein the filter is moveable to a position between the X-ray source and the tissue when the X-ray source is irradiating the tissue to modify the energy spectrum and irradiance of the initial X-ray beam.

In an embodiment of the control system for an in vivo tissue imaging system disclosed above, the configuration data further comprises at least one of timing data, location data, or orientation data of the tissue, wherein the tissue is moveable relative to the imaging system.

In an embodiment of the control system for an in vivo tissue imaging system disclosed above, at least one of the coded aperture, the X-ray source, the collimator, or a plurality of the X-ray detecting elements is moveable and controllable by the processor, wherein the configuration data further comprises at least one of timing data, location data, or orientation data for the at least one moveable X-ray source, collimator, coded aperture, or plurality of X-ray detecting elements.

In an embodiment of the control system for an in vivo tissue imaging system disclosed above, at least one of the X-ray source or a plurality of the X-ray detecting elements is moveable and controllable by the processor, wherein the configuration data further comprises at least one of timing data, location data, or orientation data for the at least one moveable X-ray source or plurality of X-ray detecting elements, and wherein the at least one moveable X-ray source or plurality of X-ray detecting elements is operable while in motion.

In an embodiment of the control system for an in vivo tissue imaging system disclosed above, at least one of the X-ray source, collimator, coded aperture, or plurality of X-ray detecting elements is configurable to enable a plurality of different perspectives of the tissue for the X-ray scatter measurement, and the processor is further configured for analyzing received X-ray scatter data from a plurality of different perspectives of the tissue to generate the representation of the irradiated tissue.

In an embodiment of the control system for an in vivo tissue imaging system disclosed above, the processor is further configured for computing an estimate of a data quality metric for the resulting X-ray scatter data from the configuration data. The processor is further configured for computing optimized configuration data using the estimated X-ray scatter data quality metric, wherein the optimized configuration data is computed to define a perspective for the X-ray scatter measurement of a point in the tissue to satisfy at least one of minimizing the attenuation of the initial X-ray beam along the initial X-ray beam path to the point in the tissue or minimizing the attenuation of the scattered X-ray signal from the point in the tissue to an X-ray detecting element.

In an embodiment of the control system for an in vivo tissue imaging system disclosed above, the processor is further configured for computing an estimate of radiation dose to the patient for the X-ray scatter measurement from the configuration data, computing an estimate of a data quality metric for the resulting X-ray scatter data from the configuration data, computing optimized configuration data using the estimated radiation dose and the estimated X-ray scatter data quality metric, and configuring the imaging system for the X-ray scatter measurement based on the computed configuration data.

In an embodiment of the control system for an in vivo tissue imaging system disclosed above, the processor is further configured for receiving recording data from a camera that records the patient, analyzing the recording data from the camera to compute an estimate of patient motion, and using the computed estimate of patient motion in combination with received X-ray scatter data to generate a representation of the irradiated tissue that accounts for an effect of patient motion.

In an embodiment of the control system for an in vivo tissue imaging system disclosed above, the processor is further configured for analyzing the estimate of patient motion to compute configuration data for the X-ray source, collimator, coded aperture, or plurality of X-ray detecting elements. The configuration data is computed to minimize an effect of patient motion on the X-ray scatter data. The processor is further configured for configuring the imaging system for the X-ray scatter measurement based on the computed configuration data.

In an embodiment of the control system for an in vivo tissue imaging system disclosed above, the coded aperture includes a periodic pattern in at least one dimension having a single attenuating component with openings focused at a millimeter-scale focal point 100-2000 millimeters from the coded aperture, the coded aperture is moveable and controllable by the processor, the tissue to be irradiated is moveable and controllable by the processor, and the configuration data further comprises at least one of timing data for the tissue, location data for the tissue, orientation data for the tissue, timing data for the coded aperture, location data for the coded aperture, or orientation data for the coded aperture to direct the focal point of the coded aperture at a point in the tissue.

In an embodiment of the control system for an in vivo tissue imaging system disclosed above, the coded aperture comprises a set of moveable and controllable attenuating components arranged in a periodic pattern in at least one dimension, the tissue to be irradiated is moveable and controllable by the processor, and the configuration data further comprises at least one of timing data for the tissue, location data for the tissue, orientation data for the tissue, timing data for the attenuating components, location data for the attenuating components, or orientation data for the attenuating components to focus the coded aperture at a millimeter-scale focal point 100-2000 millimeters from the coded aperture and direct the focal point at a point in the tissue.

In an embodiment of the control system for an in vivo tissue imaging system disclosed above, the processor is further configured for transmitting the representation of the irradiated tissue to a display.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium (including, but not limited to, non-transitory computer readable storage media). A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter situation scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer. special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

In certain embodiments, the invention may be coupled to multiple displays, printers, workstations, and/or similar devices located either locally or remotely, for example, within an institution or hospital, or in an entirely different location via one or more configurable wired and/or wireless networks such as the Internet and/or virtual private networks, wireless telephone networks, wireless local area networks, wired local area networks, wireless wide area networks, wired wide area networks, etc. The embodiments can be coupled to a picture archiving and communications system (PACS).

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The terminology used herein is for the purpose of describing particular embodiments only and is not intended to be limiting of the invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A spatially resolved volumetric tissue imaging system for performing in vivo imaging of a patient, the imaging system comprising:
an enclosure comprising a bore and a gantry positioned around the bore, wherein the bore is configured to receive at least a portion of the patient along a bore axis;
an X-ray source for irradiating a tissue volume of the at least a portion of the patient with a primary X-ray beam,
wherein the X-ray source is mounted to the gantry,
wherein the X-ray source is configurable to change an orientation of the primary X-ray beam about the bore axis and an exposure time;
a collimator positioned between the X-ray source and the at least a portion of the patient along the bore axis to shape the primary X-ray beam;
an X-ray detector array comprising a plurality of X-ray detecting elements in at least two dimensions,
wherein at least one of the plurality of the X-ray detecting elements are positioned distally from the X-ray source outside a path of the primary X-ray beam past the irradiated at least a portion of the patient to measure scattered X-ray radiation from the primary X-ray beam passing through the tissue volume;
a coded aperture positioned between the at least a portion of the patient along the bore axis and the X-ray detector array,
wherein the coded aperture is configured to modulate the scattered X-ray radiation from the tissue volume detected by the X-ray detector array; and
a control system comprising memory and a processor, wherein the processor is configured for:
configuring the imaging system for performing an X-ray scatter measurement based on configuration data comprising the orientation of the primary beam relative to the bore axis and exposure time for the X-ray source;
performing the X-ray scatter measurement with the configured imaging system;
receiving data representing scattered X-ray radiation detected by the X-ray detector array; and
estimating a spatially resolved X-ray scatter spectral reconstruction of the tissue based on the received X-ray scatter data and the configuration data;
wherein a spatially resolved tissue property or an image thereof is determined based on the estimated spatially resolved X-ray scatter spectral reconstruction of the tissue volume.

2. The imaging system of claim 1,
wherein at least one of the plurality of X-ray detecting elements are positioned to detect X-rays transmitted directly through the tissue volume from the primary X-ray beam such that the imaging system operates as an X-ray transmission imaging system, and
wherein the processor is further configured for:
configuring the imaging system for performing an X-ray transmission measurement based on the configuration data,
wherein the configuration data further comprises the orientation of the primary beam relative to the bore axis and the exposure time for the X-ray transmission measurement;
performing the X-ray transmission measurement with the configured imaging system;
receiving data representing transmitted X-ray radiation detected by the X-ray detector array; and
computing an X-ray radiodensity tissue reconstruction from the received X-ray transmission data.

3-12. (canceled)

13. The imaging system of claim 1, wherein the coded aperture is mounted to the gantry or positioned in the bore.

14. (canceled)

15. The imaging system of claim 1, wherein the at least one of the plurality of X-ray detecting elements positioned distally from the X-ray source to detect the scattered X-ray radiation is maintained at a location along the bore axis in the imaging system such that the primary X-ray beam does not impinge on the at least one of the plurality of X-ray detecting elements positioned to detect the scattered X-ray radiation during the scatter measurement.

16-18. (canceled)

19. The imaging system of claim 2,
wherein the coded aperture is moveable to a position between the at least a portion of the patient along the bore axis and the X-ray detector array to modulate the scattered X-ray radiation during the X-ray scatter measurement,
wherein a coded aperture movement is controllable by the processor, and
wherein the configuration data further comprises at least one of timing data, location data, or orientation data for the coded aperture.

20. The imaging system of claim 2,
wherein at least one of the X-ray source, the collimator, or at least one of the plurality of X-ray detecting elements is moveable and controllable by the processor, and
wherein the configuration data further comprises at least one of timing data, location data, or orientation data for the at least one moveable X-ray source, collimator, or the at least one of the plurality of X-ray detecting elements such that the at least one of the plurality of the X-ray detecting elements is offset at an angle from the primary X-ray beam path for an X-ray scatter measurement to increase a relative solid angular coverage of the X-ray detector array for a range of X-ray scatter angles from a point in the tissue volume.

21. The imaging system of claim 2, wherein the processor is further configured for:
identifying a region of interest in the patient based on the X-ray radiodensity tissue reconstruction;
computing configuration data for the X-ray scatter measurement based on the identified region of interest in the patient; and
configuring the imaging system for the X-ray scatter measurement based on the computed configuration data.

22-23. (canceled)

24. The imaging system of claim 21, wherein the processor is further configured for:
computing a spatially resolved estimate of a tissue property from the X-ray radiodensity tissue reconstruction; and
computing a region of interest in the X-ray radiodensity tissue reconstruction using the spatially resolved estimate of the tissue property.

25-27. (canceled)

28. The imaging system of claim 1, wherein the processor is further configured for reconstructing an estimate of spatially resolved X-ray scatter spectra of the irradiated tissue volume using a received X-ray scatter data and a forward model of the imaging system.

29. (canceled)

30. The imaging system of claim 1, wherein the processor is further configured for:
computing a spatially resolved estimate of a momentum transfer spectra of the irradiated tissue from the X-ray scatter data.

31-35. (canceled)

36. The imaging system of claim 1, wherein the collimator includes an opening that is configurable in at least one dimension to shape the primary X-ray beam, wherein the at least one dimension of the collimator opening is controllable by the processor, and wherein the configuration data further comprises a dimension of the opening of the collimator.

37. (canceled)

38. (canceled)

39. The imaging system of claim 1, wherein at least one of the coded aperture, the X-ray source, the collimator, or at least one of the plurality of X-ray detecting elements is moveable and controllable by the processor, wherein the configuration data further comprises at least one of timing data, location data, or orientation data for the at least one moveable X-ray source, the collimator, the coded aperture, or the at least one of the plurality of X-ray detecting elements.

40. (canceled)

41. The imaging system of claim 1,
wherein the configuration data for the X-ray scatter measurement further comprises more than one orientation of the primary X-ray beam about the bore axis,
wherein the X-ray scatter measurement comprises measuring scattered X-ray radiation from the primary X-ray beam passing through the tissue volume from more than one orientation about the bore axis, and
wherein the processor is further configured for estimating the spatially resolved X-ray scatter spectral reconstruction of the tissue volume based on the received X-ray scatter data from more than one perspective of the tissue volume.

42. (canceled)

43. The imaging system of claim 1, wherein the processor is further configured for:
computing an estimate of radiation dose to the patient for an X-ray scatter measurement from the configuration data;
computing an estimate of a data quality metric for a resulting X-ray scatter data from the configuration data; and
computing optimized configuration data using the estimated radiation dose and the estimation of the X-ray scatter data quality metric.

44. (canceled)

45. (canceled)

46. The imaging system of claim 1,
wherein the coded aperture comprises a single attenuating component with a pattern of openings in at least one dimension, with the openings focused at a millimeter-scale focal point between the coded aperture and the X-ray source,
wherein the coded aperture is moveable and controllable by the processor,
wherein the tissue volume to be irradiated is moveable and controllable by the processor, and wherein the configuration data further comprises at least one of timing data for the tissue volume, location data for the tissue volume, orientation data for the tissue volume, timing data for the coded aperture, location data for the coded aperture, or orientation data for the coded aperture to direct the focal point of the coded aperture at a point in the tissue volume.

47. The imaging system of claim 1,
wherein the coded aperture comprises a set of moveable and controllable attenuating components arranged in a pattern in at least one dimension,
wherein the tissue volume to be irradiated is moveable and controllable by the processor, and
wherein the configuration data further comprises at least one of timing data for the tissue volume, location data for the tissue, orientation data for the tissue volume, timing data for the attenuating components, location data for the attenuating components, or orientation data for the attenuating components to focus the coded aperture at a millimeter-scale focal point between the coded aperture and the X-ray source and direct the focal point at a point in the tissue volume.

48. (canceled)

49. (canceled)

50. An imaging system for performing in vivo imaging of a patient, the imaging system comprising:
an X-ray source mounted to a configurable arm for irradiating a tissue volume of at least a portion of the patient with a primary X-ray beam,
wherein a position or an orientation of the X-ray source is adjustable by a user;
a collimator positioned between the X-ray source and the at least a portion of the patient to shape the primary X-ray beam;
an X-ray detector array comprising a plurality of X-ray detecting elements arranged in at least two dimensions,
wherein at least one of the plurality of X-ray detecting elements is positioned distally from the X-ray source outside a path of the primary X-ray beam past the irradiated at least a portion of the patient to measure scattered X-ray radiation from the primary X-ray beam passing through the tissue volume;
a coded aperture positioned between the at least a portion of the tissue and the X-ray detector array, wherein the coded aperture is configured to modulate the scattered X-ray radiation from the tissue volume detected by the X-ray detector array; and
a control system comprising memory and a processor, wherein the processor is configured for:
determining configuration data for the imaging system,
wherein the configuration data comprises a location or an orientation of the X-ray source;
performing the X-ray scatter measurement with the configured imaging system;
receiving data representing scattered X-ray radiation detected by the X-ray detector array; and
estimating a spatially resolved X-ray scatter spectral reconstruction of the tissue based on the received X-ray scatter data and the configuration data;
wherein a spatially resolved tissue property or an image thereof is determined based on the estimated spatially resolved X-ray scatter spectral reconstruction of the tissue volume.

51. The imaging system of claim 50,
wherein the configurable arm is a C-arm with the X-ray source mounted proximate to a first end of the C-arm and at least one of the plurality of X-ray detecting elements mounted proximate to a second end of the C-arm,
wherein the C-arm is adjustable by the user such that a least a portion of the patient is positioned between the X-ray source and the at least one of the plurality of X-ray detecting element for the X-ray scatter measurement.

52. The imaging system of claim 50, wherein the configurable arm is mounted to a ceiling, floor, wall, or other fixed surface.

53. The imaging system of claim 50, wherein the configurable arm is mounted to a mobile carriage, wherein the carriage can be positioned by the user.

54. (canceled)

55. (canceled)

56. A method for performing in vivo tissue imaging of a patient, the method comprising:
positioning at least a portion of the patient in an imaging system;
configuring the imaging system for an X-ray scatter measurement based on configuration data comprising an orientation of a primary X-ray beam relative to a bore axis and exposure time for an X-ray source;
performing the X-ray scatter measurement with the configured imaging system, wherein the X-ray scatter measurement comprises:
irradiating a tissue volume of at least a portion of the patient with a primary X-ray beam from the X-ray source through a collimator positioned between the X-ray source and the at least a portion of the tissue to shape the primary X-ray beam;
modulating scattered X-ray radiation from the tissue using a coded aperture positioned between the at least a portion of the tissue and an X-ray detector array comprising a plurality of X-ray detecting elements in at least two dimensions;
detecting the modulated scattered X-ray radiation from the tissue volume with at least one of the plurality of X-ray detecting elements positioned distally from the X-ray source outside a path of the primary X-ray beam past the irradiated at least a portion of the patient to measure scattered X-ray radiation from the primary X-ray beam passing through the tissue volume; and
receiving data representing the detected scattered X-ray radiation from the X-ray detector array; and
estimating a spatially resolved X-ray scatter spectral reconstruction of the tissue volume based on the received X-ray scatter data and the configuration data;
wherein a spatially resolved tissue property or an image thereof is determined based on the estimated spatially resolved X-ray scatter spectral reconstruction of the tissue volume.

57-104. (canceled)

105. The imaging system of claim 1, wherein the processor of the control system is further configured for: determining a spatially resolved tissue property based on the received X-ray scatter data.

106. The imaging system of claim 1, wherein the processor of the control system is further configured for: producing a spatially resolved scatter tissue image based on the received X-ray scatter data.
